(19) United States
(12) Patent Application Publication
(10) Pub. No.: US 2024/0373849 A1
(43) Pub. Date: Nov. 14, 2024

(54) INSECT NEUROPEPTIDE ANALOGUES

(71) Applicant: THE UNIVERSITY COURT OF THE UNIVERSITY OF GLASGOW, Glasgow (GB)

(72) Inventors: Shireen A. Davies, Glasgow (GB); Robert M.J. Liskamp, Glasgow (GB); Julian A.T. Dow, Glasgow (GB)

(73) Assignee: THE UNIVERSITY COURT OF THE UNIVERSITY OF GLASGOW, Glasgow (GB)

(21) Appl. No.: 18/007,513

(22) PCT Filed: Jun. 4, 2021
(86) PCT No.: PCT/GB2021/051401

§ 371 Date: Dec. 1, 2022

(30) Foreign Application Priority Data
Jun. 4, 2020 (GB) 2008445.5

(51) Int. Cl. A01N 37/46; A01N 47/44; A01P 7/04; C07K 14/435

(52) U.S. Cl. CPC A01N 37/46; A01N 47/44; A01P 7/04; C07K 14/43563

(57) ABSTRACT

The present invention relates to analogues of insect neuropeptides having activity against hemipteran and dipteran insects, such as aphids and fruit flies, and their use as insect control agents (e.g. insecticides) and plant protection agents.

### FIELD OF THE INVENTION

The present invention relates to analogues of insect neuropeptides having activity against hemipteran and dipteran insects, such as aphids and fruit flies, and their use as insect control agents (e.g. insecticides) and plant protection agents.

### BACKGROUND

With a global dependence on broad-spectrum insecticides, the damaging effects of which are well documented, there is increasing need for the development of greener, target-specific insecticides. The development and employment of neuropeptide synthetic analogues offers a promising avenue in the drive for greener and target-specific insecticidal agents. Within the insects, neuropeptides are regulatory peptides with functional roles in growth and development, behaviour and reproduction, metabolism and homeostasis, and muscle movement. Due to their high specificity, neuropeptides and their cognate receptors (G-protein coupled receptors, GPCRs) may be developed towards insecticidal agents to selectively reduce the fitness of target pest insects, whilst minimising detrimental environmental impacts.

Insect neuropeptide families include the insect kinins and cardio acceleratory peptides (CAPA, CAP2b) neuropeptides.

The CAPA peptides, were first identified from the moth Manduca sexta (CAP2b) and have since been identified in many insect families. Although function varies depending on insect species, life stage, and lifestyle, CAPA peptides play a key role in myomodulation and osmoregulation¹⁶ and have more recently been linked to desiccation and cold tolerance in Drosophila species.

The CAPA peptides belong to the PRXamide superfamily which can be further subdivided into three major classes: CAPA peptides, pyrokinins (PK) and ecdysis triggering hormone (ETH). The pyrokinins are further subdivided into diapause hormone (DH) and pheromone biosynthesis activating neuropeptides (PBAN) and by their C-terminal motifs WFGPRLamide and FXPRLamide respectively.

### SUMMARY OF THE INVENTION

The inventors have discovered new analogues of CAPA peptides having insecticidal activity against hemipteran and/or dipteran insects, and so potentially finding use as pest control agents or insecticides, while having little or no effect against important pollinator species such as bees.

Thus, in a first aspect, the invention provides an insecticidal compound having the formula:

R¹-L¹-Z-R²

where Z is a peptide of formula:

L-X2-X3-F-X5-RV-

wherein:
X2 is V or Y;
X3 is A or Aib;
X5 is P or A; and
wherein optionally at least one of the residues in peptide Z is N-methylated;
L¹ is absent or a residue of any amino acid, e.g. *—(C═O)C₁₋₁₀-alkylene-NH— where * denotes the point of attachment to Z;
R¹ is hydrogen (which may be designated “H—” or “Hy-”), C₁₋₄ alkyl (e.g. methyl, ethyl, propyl, butyl), —N(R^{1a})—C(═N⁺(R^{1a})₂)NR^{1a}₂, —C(═N⁺(R^{1a}))NR^{1a}₂, acyl, fatty acyl, benzyl, or benzoyl; wherein each R^{1a} is independently selected from hydrogen or C₁₋₄ alkyl (e.g. methyl, ethyl, propyl, butyl); and
R² is NH₂, NR^{2a}H, NR^{2a}₂, or OR^{2a} wherein each R^{2a} if present is independently C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkylene-C₆₋₁₆-aryl, or C₁₋₆-haloalkyl, each of which may optionally be substituted with one or more groups selected from halogen, C₁₋₆-alkyl, or C₁₋₆-haloalkyl.

In some embodiments L¹ is absent or is *—(C═O)C₁₋₁₀-alkylene-NH— where * denotes the point of attachment to Z, e.g. L¹ is *—(C═O)C₁₋₆-alkylene-NH— such as

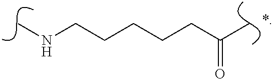

In some embodiments, therefore, R¹ is hydrogen (which may be designated “H—” or “Hy-”), C₁₋₄ alkyl (e.g. methyl, ethyl, propyl, butyl), —N(R^{1a})—C(═N⁺(R^{1a})₂)NR^{1a}₂, or —C(═N⁺(R^{1a})₂)NR^{1a}₂; wherein each R^{1a} is independently selected from hydrogen or C₁₋₄ alkyl (e.g. methyl, ethyl, propyl, butyl).

In some embodiments R¹ is hydrogen or —C(═N⁺(R^{1a})₂)NR^{1a}₂ such as —C(═N⁺Me₂)NMe₂.

In some embodiments R² is NH₂.

In some embodiments when R¹ is hydrogen, L¹ is absent or *—(C═O)C₁₋₁₀-alkylene-NH— where * denotes the point of attachment to Z, e.g. R¹ is hydrogen and L¹ is *—(C═O)C₁₋₆-alkylene-NH— such as

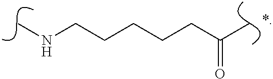

In some embodiments, when R¹ is —C(═N⁺(R^{1a})₂)NR^{1a}₂, L¹ is absent e.g. R¹ is —C(═N⁺Me₂)NMe₂ and L¹ is absent. In such embodiments the R¹ group forms the following guanidine based structure along with the N-terminal nitrogen (denoted “N—R” in the structure below where R is H or Methyl) of the peptide sequence Z:

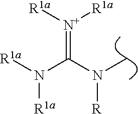

In some embodiments, X2 is V and/or X5 is P, e.g. X2 is V and X5 is P. Such compounds may be considered analogues of CAPA2.

In such embodiments, examples of peptide Z include:

(SEQ ID NO: 1)
LVAFPRV;
(SEQ ID NO: 2)
LVAFPR-(Me)V;
(SEQ ID NO: 3)
L-(Me)V-AFPRV;
(SEQ ID NO: 41)
LV-(Me)AFPRV;
and
(SEQ ID NO: 4)
LV-(Me)A-FPR-(Me)V.

In some embodiments, L¹ is absent or *—(C═O)C₁₋₁₀-alkylene-NH— where * denotes the point of attachment to Z, e.g. L¹ is *—(C═O)C₁₋₆-alkylene-NH— such as

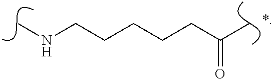

In some embodiments, R¹ is H or —C(═N⁺(R^{1a})₂)NR^{1a}₂ such as —C(═N⁺Me₂)NMe₂.

Typically, when L¹ is —(C═O)C₁₋₁₀-alkylene-NH—, R¹ is H.

Typically when R¹ is —C(═N⁺(R^{1a})₂)NR^{1a}₂, L¹ is absent. When R¹ is “—C(═N⁺(R^{1a})₂)NR^{1a}₂” and L¹ is absent, the R¹ group together with the N-terminal nitrogen of the peptide sequence Z form a guanidine based group (discussed above). Preferably —C(═N⁺(R^{1a})₂)NR^{1a}₂ is —C(═N⁺Me₂)NMe₂.

In some embodiments, R² is NH₂, NR^{2a}H or NR^{2a}₂. Preferably R² is NH₂.

Examples of CAPA2 analogue peptides include:

(AH56)
(SEQ ID NO: 5)
H-Ahx-LVAFPRV-NH₂;
(AH257)
(SEQ ID NO: 6)
Guanidyl-LVAFPR-(Me)V-NH₂;
(AH259)
(SEQ ID NO: 7)
Guanidyl-L-(Me)V-AFPRV-NH₂;
(AH283)
(SEQ ID NO: 8)
H-Ahx-L-(Me)V-AFPRV-NH₂
(AH283a)
(SEQ ID NO: 9)
H-Ahx-LVAFPR-(Me)V-NH₂;
(AH270)
(SEQ ID NO: 10)
H-Ahx-LV-(Me)A-FPR-(Me)V-NH₂
(AH258)
SEQ ID NO: 11)
Guanidyl-LV-(Me)A-FPRV-NH₂
(AH59)
(SEQ ID NO: 12)
Palmitoyl-LVAFPRV-NH₂
(AH55)
(SEQ ID NO: 13)
4-Benzoyl benzoic-LVAFPRV-NH₂
and
(AH33)
(SEQ ID NO: 14)
Ac-LVAFPRV-NH₂

Examples of CAPA2 analogue peptides include:

(AH56)
(SEQ ID NO: 5)
H-Ahx-LVAFPRV-NH₂;
(AH257)
(SEQ ID NO: 6)
Guanidyl-LVAFPR-(Me)V-NH₂;
(AH259)
(SEQ ID NO: 7)
Guanidyl-L-(Me)V-AFPRV-NH₂;
(AH283)
(SEQ ID NO: 8)
H-Ahx-L-(Me)V-AFPRV-NH₂
(AH283a)
(SEQ ID NO: 9)
H-Ahx-LVAFPR-(Me)V-NH₂;
and
(AH270)
(SEQ ID NO: 10)
H-Ahx-LV-(Me)A-FPR-(Me)V-NH₂

In one embodiment, the compound of the invention may be any of the CAPA2 analogues listed herein.

“Guanidyl” refers to the case where the R¹ is —C(═N⁺Me₂)NMe₂ and the terminal structure formed by “Guanidyl-L-” is as follows:

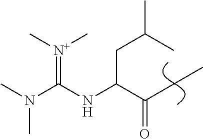 [SMILES: CCC(=O)C(CC(C)C)NC(N(C)C)=[N+](C)C]

In some embodiments, X2 is Y and/or X5 is A, e.g. X2 is Y and X5 is A. Such compounds may be considered analogues of CAPA1.

In such embodiments, examples of peptide Z include:

(SEQ ID NO: 15)
LYAFARV;
(SEQ ID NO: 16)
LYAFAR-(Me)V;
(SEQ ID NO: 17)
LYAF-(Me)A-RV;
(SEQ ID NO: 18)
(Me)L-YAFARV;
and
(SEQ ID NO: 19)
LY-Aib-FARV.

In some embodiments, L¹ is absent.

In some embodiments, R¹ is H.

In some embodiments, R² is NH₂, NR^{2a}H, or NR^{2a}₂. Preferably R² is NH₂.

Examples of CAPA1 analogue peptides include:

(AH188)
(SEQ ID NO: 20)
H-LYAFARV-NH₂;
(AH380)
(SEQ ID NO: 21)
H-LYAFAR-(Me)V-NH₂;
(AH382)
(SEQ ID NO: 22)
H-LY-(Me)A-FARV-NH₂
(AH382a)
(SEQ ID NO: 23)
H-LYAF-(Me)A-RV-NH₂;
(AH383)
(SEQ ID NO: 24)
H-(Me)L-YAFARV-HN₂;
(AH387)
(SEQ ID NO: 25)
H-LY-Aib-FARV-NH₂
(AH49)
(SEQ ID NO: 26)
4-Benzoyl benzoic-LYAFPRV-NH₂
(AH32)
(SEQ ID NO: 27)
Ac-LYAFPRV-NH₂
(AH62)
(SEQ ID NO: 28)
Palmitoyl-LYAFPRV-NH₂
and
(AH51)
(SEQ ID NO: 29)
H-Nle-LYAFPRV-NH₂

Examples of CAPA1 analogue peptides include:

(AH188)
(SEQ ID NO: 20)
H-LYAFARV-NH₂;
(AH380)
(SEQ ID NO: 21)
H-LYAFAR-(Me)V-NH₂;
(AH382)
(SEQ ID NO: 22)
H-LY-(Me)A-FARV-NH₂
(AH382a)
(SEQ ID NO: 23)
H-LYAF-(Me)A-RV-NH₂;
(AH383)
(SEQ ID NO: 24)
H-(Me)L-YAFARV-HN₂;
and
(AH387)
(SEQ ID NO: 25)
H-LY-Aib-FARV-NH₂.

In one embodiment, the compound of the invention may be any of the CAPA1 analogues listed herein.

In some embodiments, the compounds may be modified, suitably modified at the N or C terminus. Suitably, the compounds may be modified to increase cuticle permeability or to increase stability. Suitably, the compound may be modified with an aromatic, aliphatic or lipophilic group. Suitably, R¹ may be an aromatic, aliphatic or lipophilic group.

In one embodiment, the compound may be modified with a lipophilic group such as a fatty acid derivative, for example a fatty acyl group such as palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl. Suitably therefore the R¹ group may be a fatty acyl group such as palmitoyl.

In one embodiment, the compound may be modified with an aromatic group such as a benzyl or benzoyl group which may be a benzoic acid derivative or benzophenone derivative. Suitably therefore the R¹ group may be an aromatic group such as 4-benzoyl benzoic acid, or a derivative such as 4-benzoyl benzoyl.

In one embodiment, the compound may be modified with an acyl group i.e. an R_{1b}—C(O)— group wherein R^{1b} is a C1-C4 alkyl, for example formyl, acetyl (Ac), propanoyl, butanoyl, or wherein R^{1b} is benzoyl. Suitably therefore the R¹ group may be R^{1b}—C(O)— such as acetyl (Ac).

Suitably the compound may also be modified with a polymer. Suitably the R¹ group may be a polymer. Suitably the polymer may increase the ease of formulation of the compound. In one embodiment, the compound may be PEGylated, suitably by covalent attachment of polyethylene glycol. In one embodiment, the compound may comprise PEG-Ahx-LV-(Me)A-FPR-(Me)V-NH₂ (PEGAH270/AHPEG270) (SEQ ID NO: 30).

In some embodiments, the compounds of the invention may be salts thereof.

The compounds have activity against hemipteran insects and/or dipteran insects.

The compounds typically increase insect mortality, for example when contacted topically to a suitable insect, or ingested by a suitable insect. Thus, the compounds described (and compositions containing them) may be regarded as insecticides, and may be referred to as “insect control agents”.

Without wishing to be bound by theory, any or all of the effects described may be mediated by agonist activity at the CapaR receptor of the target insects. The CapaR receptor of Drosophila melanogaster may be used as a model system, as described in the examples below. Agonist activity may be assessed by any suitable read-out, such as an increase in intracellular calcium. The term “Capa” is now in more common use than the previously-used term “CAP2b”. The terms “CAP2b” and “Capa” may be used interchangeably, as may “CAP2b receptor” and “Capa receptor”.

It is believed that, inter alia, the analogues described in this specification retain agonist activity while having superior stability compared to wild type Capa peptides, especially against proteases. Consequently, they are believed to have superior applicability as insecticides.

The invention provides a method of increasing insect mortality, comprising contacting an insect or insect population with a compound as described. The insect or insect population may be hemipteran and/or dipteran.

The invention further provides a method of decreasing insect feeding, comprising contacting an insect or insect population with a compound as described. Suitably decreasing insect feeding on a plant or plant part. The insect or insect population may be hemipteran and/or dipteran.

The compound may be applied directly to an insect or insect population. For example, it may be applied topically. Alternatively, the compound may be applied indirectly. For example, it may be applied to a substrate likely to come into contact with an insect or insect population. The substrate may be a plant or plant part, especially for Hemiptera or Diptera which represent pests of plants (whether crops or horticultural plants).

However, for insects which represent pests to humans, such as the Cimicidae family (e.g. bedbugs of the genus Cimex, such as Cimex lectularius) or the Reduviidae family (e.g. of the genus Rhodnius such as Rhodnius prolixus, or Triatoma such as Triatoma infestans) which can be vectors of human disease, the substrate may be a domestic surface or article, such as bedding, a mattress, or any other suitable domestic surface. The compound may be applied to the substrate in a form suitable for ingestion by an insect.

The invention further provides the use of a compound as described as a plant protection agent, and specifically for protecting a plant or plant part against hemipteran and/or dipteran insects.

The invention further provides a method of inhibiting infestation of a plant or plant part by hemipteran and/or dipteran insects comprising contacting the plant or plant part with a compound as described.

The method may be prophylactic. Thus, for example, the compound may be applied to the plant or plant part while the plant or part is free or substantially free of hemipteran and/or dipteran insects.

Alternatively, the plant or plant part may already be colonised or infested by hemipteran and/or dipteran insects. Thus, the invention further provides a method of reducing infestation of a plant or plant part, or of reducing hemipteran and/or dipteran insect load on a plant or plant part, the method comprising contacting the plant or plant part with a compound as described.

In any of these embodiments, the compound may be provided as part of a composition, such as an insect control composition (e.g. insecticide composition) or a plant protection composition. Reference to application or use of a compound should therefore be construed as encompassing application or use of a suitable composition, unless the context demands otherwise.

The composition typically comprises a compound as described in combination with one or more ancillary component such as solvents, carriers, diluents, adjuvants, preservatives, dispersants, emulsifying agents, or synergists.

The composition may further comprise one or more additional active insecticides.

The invention further provides a composition, e.g. an insect control composition or plant protection composition, comprising a compound of the invention in admixture with one or more solvents, carriers, diluents, adjuvants, preservatives, dispersants, emulsifying agents, or synergists. The composition may be an aqueous composition.

The invention includes the combination of the aspects and preferred features described except where such a combination is impermissible or expressly avoided.

### BRIEF DESCRIPTION OF THE FIGURES

FIG. 1. Results of Drosophila suzukii feeding assay

Results are shown as % lethality in the population, after correction for baseline. Statistical test (P<0.05) Unpaired T-Test with Welch's correction, significance compared to scrambled capa 1 and 2 control and no peptide control (panel B); or no peptide control (panel A).

FIG. 2. Results of Honeydew Production Assay

FIG. 2A shows the honeydew production from control aphids compared to FIG. 2B which shows reduced honeydew production in peptide-fed aphids. Purple staining indicates honeydew production and therefore aphid feeding.

FIG. 3. Peptide efficacies against Green Peach Aphid, via feeding

Results are shown as % lethality in the population. FIG. 3A shows results for peptide AH383. FIG. 3B shows results for peptide AH387. FIG. 3C shows results for peptide AH270 and PEGAH270 (a pegylated form of AH270).

FIG. 4. Peptide efficacies against Green Peach Aphid, via spraying (Potter Tower)

Results are shown as % lethality in the population of aphids per plant. Efficacy data is from foliar spray application of peptides to plants infested with Green Peach Aphids using a Potter Tower, each dot is a plant infested with 30 aphids. Imidacloprid is a positive control.

FIG. 5. Peptide efficacies against Green Peach Aphid on Treated Leaf Surfaces

Results are shown as % lethality over total treated leaf area. Efficacy data is from foliar spray application of peptides to individual leaves, then infested with Green Peach Aphids. AH270 Modified is a pegylated form of AH270. Spirotetramat is a positive control.

FIG. 6. Activation of D. suzukii Capa Receptors by Peptides

(A) activation of D. suzukii CapaR receptors by endogenous capa peptide; and (B) differential activation of D. suzukii CapaR receptors by different peptide candidates.

FIG. 7. Peptide efficacies against D. suzukii larvae, via feeding

In vivo efficacies data shown after 72 hour treatment with indicated peptides A-F. AH270/PEGAH270 treatments were 120 h. Median values are indicated. Statistical analysis (Welch t-test) indicate statistically significant mean efficacy compared to control (p<0.0001 for all peptides apart from AH56, p<0.01).

FIG. 8. Dose-response curves for Peptides in D. suzukii

Dose response experiments were carried out for AH382, AH383, AH188, data shown for AH382 (a), AH383 (b), AH188 (c, 48 hours). Data are mean % lethality ±SEM for concentrations between 10⁻⁴ M and 10⁻⁷ M (a) AH382, (b) AH383 72 hours treatment; and between 10⁻⁵ M and 10⁻⁹ M (AH188, 3c), 48 hours treatment. The LD50 for AH382, AH383 and AH188 is 10⁻⁶ M.

FIG. 9. Assessment of in vivo efficacy of 1st and later generation peptides by % lethality

Summary of data gathered from D. suzukii larval feeding assays.

FIG. 10. Comparison of peptide targeting of different species

Endogenous Capa peptides comprising FPRV motif target and bind to the intended target species 1 and 2 of aphids and Drosophila such as D. suzukii for example, but do not target unintended species such as bumblebees.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Definitions

Throughout the present description and claims the conventional three-letter and one-letter codes for naturally occurring amino acids are used, i.e.

A (Ala), G (Gly), L (Leu), I (Ile), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro).

By “naturally occurring” in this context is meant the 20 amino acids encoded by the standard genetic code, sometimes referred to as proteinogenic amino acids.

Generally accepted three-letter codes and other abbreviations for other amino acids may also be employed, such as hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib), etc. Ahx indicates 6-aminohexanoic acid (also known as 6-aminocaproic acid or F-aminocaproic acid). ‘amino acid’ as referred to herein may refer to a naturally occurring amino acid or any other amino acid including synthetic amino acids, and non-proteinogenic amino acids.

The notation “(Me)” before an amino acid code is used to indicate an N-methylated amino acid residue. Thus, for example, “(Me)V” indicates N-methyl valine, “(Me)A” indicates N-methyl alanine and “(Me)L” indicates N-methyl leucine.

Such other amino acids may be shown in square brackets “[ ]” (e.g. “[Aib]”) when used in a general formula or sequence in the present specification, especially when the rest of the formula or sequence is shown using the single letter code.

Unless otherwise specified, amino acid residues in peptides of the invention are of the L-configuration. However, D-configuration amino acids may be incorporated. In the present context, an amino acid code written with a small letter may be used to represent the D-configuration of said amino acid.

The notation Cₓ₋ₓₓrefers to the number of carbon atoms in a functional group. The number in the ‘x’ positions is the lowest number of carbon atoms and the number in the ‘xx’ position denotes the highest number of carbon atoms. For example, C₁₋₆-alkyl refers to alkyl groups as defined herein having from 1 to 6 carbon atoms.

The notation i, n or t are used herein in relation to various alkyl groups in the normal way. Specifically, the suffixes refer to the arrangement of atoms and denotes straight chain (‘n’) or branched (‘i’ or ‘t’) alkyl groups.

The term alkyl as used herein refers to a saturated linear or branched-chain monovalent hydrocarbon radical, wherein the alkyl radical may be optionally substituted. The number of carbon atoms in the alkyl group may be specified using the above notation, for example, when there are from 1 to 8 carbon atoms the term “C₁₋₈-alkyl” may be used. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), and 1-butyl (n-Bu, n-butyl, —CH₂CH₂CH₂CH₃).

The term alkylene as used herein refers to a saturated, branched, or straight chain hydrocarbon group having two monovalent radical centres derived by the removal of two hydrogen atoms from the same or two different carbon atoms of a parent alkane. The number of carbon atoms in the alkylene group may be specified using the above notation, for example, when there are from 1 to 8 carbon atoms the term “C₁₋₈-alkylene” may be used. Example alkylene groups include methylene (—CH₂—), 1,1-ethylene (—CH(CH₃)—), 1,2-ethylene (—CH₂CH₂—), 1,1-propylene (—CH(CH₂CH₃)—), and 2,2-propylene (—C(CH₃)₂—).

The term alkenyl as used herein refers to a linear or branched-chain monovalent hydrocarbon radical with at least one site of unsaturation, i.e., a carbon-carbon double bond. The alkenyl radical may be optionally substituted, and includes radicals having “cis” and “trans” orientations, or alternatively, “E” and “Z” orientations. The number of carbon atoms in the alkenyl group may be specified using the above notation, for example, when there are from 2 to 8 carbon atoms the term “C₂₋₈-alkenyl” may be used. Example alkenyl groups include, but are not limited to, ethenyl (—CH═CH₂), and prop-1-enyl (—CH═CHCH₃),

In the chemical structures drawn herein, the presence of “ [symbol] ” denotes a point of attachment or a radical for example, a radical as discussed in relation to various functional groups.

The term aryl as used herein refers to a monovalent carbocyclic aromatic radical. Aryl includes groups having a single ring and groups having more than one ring such a fused rings or spirocycles. In the case of groups having more than one ring, at least one of the rings is aromatic. The number of carbon atoms in the aryl group may be specified using the above notation, for example, when there are from 6 to 16 carbon atoms the term “C₆₋₁₆-aryl” may be used. Aryl groups may be optionally substituted. Examples of aryl groups include phenyl, naphthyl, biphenyl, phenanthrenyl, naphthacenyl, 1,2,3,4-tetrahydronaphthalenyl, 1H-indenyl, 2,3-dihydro-1H-indenyl, and fluorenyl.

The term halogen as used herein refers the one or more of fluorine (F), chlorine (Cl), bromine (Br) or iodine (I).

The term haloalkyl refers to an alkyl group having on or more halogen substituent. The number of carbon atoms in the haloalkyl group may be specified using the above notation, for example, when there are from 1 to 8 carbon atoms the term “C₁₋₈-haloalkyl” may be used. Examples of haloalkyl groups include trifluoromethyl (—CF₃).

By ‘plant or plant part’, or ‘plant or part thereof’ referred to herein it is meant any part of a plant including but not limited to; the leaf, stem, root, flower, bud, bulb, and seed.

Terminal Groups R¹ and R²

The terminal groups present at the N- and C-termini of the peptide backbone are designated R¹ and R² respectively. Thus R¹ is bonded to the nitrogen atom of the N-terminal amino group (of L¹ or Z) and R² is bonded to the C-terminal carbonyl carbon atom.

R¹ is hydrogen (which may be designated “H-” or “Hy-”), C₁₋₄ alkyl (e.g. methyl, ethyl, propyl, butyl), —N(R^{1a})—C(═N⁺(R^{1a})₂)NR^{1a}₂, or —C(═N⁺(R^{1a})₂)NR^{1a}₂; wherein each R^{1a} is independently selected from hydrogen or C₁₋₄ alkyl (e.g. methyl, ethyl, propyl, butyl)

In some embodiments R¹ is hydrogen or —C(═N⁺(R^{1a})₂)NR^{1a}₂ such as —C(═N⁺Me₂)NMe₂.

R¹=“H” (or “Hy”; hydrogen) typically indicates a free primary amino group at the N-terminus. The other hydrogen atom of the N-terminal amino group is typically invariant, regardless of the nature of R¹. Exceptionally, when the residue at the N-terminus is N-methylated, R¹ may still be indicated as H even though the N-terminal residue has a secondary amine group. Thus an N-methylated leucine residue at the N-terminus may be indicated as R¹—(Me)L- where R¹ is H. However, it could also be shown as simply R¹-L- where R¹ is methyl and the other hydrogen atom is not shown.

R² is NH₂, NR^{2a}H, NR^{2A}₂, or OR^{2a}, indicating a C-terminal amido group (i.e. CONH₂, CONR^{2a}H, or CONR^{2A}₂) or ester group (COOR^{2a}). Typically, R₂ is NH₂.

L¹ group

When present, L¹ may be a residue of any amino acid, e.g. a proteinogenic amino acid.

In preferred embodiments, though, L¹ is *—(C═O)C₁₋₁₀-alkylene-NH— where * denotes the point of attachment to Z. For example, L¹ may be *—(C═O)C₁₋₆-alkylene-NH—, such as:

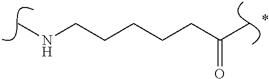

which may be regarded as a residue of 6-aminohexanoic acid (Ahx).

When L¹ is present, R¹ is typically hydrogen (H). For example, R¹ is hydrogen and L¹ is *—(C═O)C₁₋₆-alkylene-NH— such as:

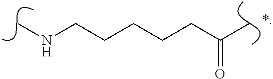

### Insect Control Agent

The term “insect control agent” refers to agents used to increase insect mortality (i.e. as insecticides). Thus an insect control agent may be administered to accelerate mortality of a given insect or insect population.

An increase in mortality used herein is intended to refer to an increase in the percentage of dead insects, as compared to the percentage of dead insects of an otherwise identical insect population which have not been exposed to the insect control agent of the invention.

Suitably, insect mortality may be calculated as number of dead insects/total number of insects per treated area. Suitably the treated area may be a well of a plate, or may be one or more leaves, or an entire plant.

An insect control agent may be used to reduce the size of an insect population, or inhibit growth of an insect population or inhibit feeding of an insect population (e.g. as compared to an otherwise identical insect population not exposed to the agent).

An insect control composition is a composition comprising an insect control agent as described.

### Plant Protection Agent

The term “plant protection agent” refers to agents when used to protect a plant or plant part against hemipteran and/or dipteran insects, e.g. against infestation or colonisation, or being used as a food source by such insects (e.g. by the draining of sap). Infestation or colonisation may be by larvae (or nymphs), by adult insects, or by being used as a host or repository for eggs. The terms “infestation” and “colonisation” should not be construed as requiring the presence of the insects to be deleterious to the plant, however.

A plant protection agent may be applied inter alia for reducing insect load on a plant or plant part, for inhibiting (e.g. reducing the rate of) increase of insect load on a plant or plant part, or for maintaining a plant in an insect-free state, as compared to an otherwise identical plant having an insect population not exposed to the agent. Thus, the agent may be applied to a plant or plant part which already carries hemipteran insects, or to a plant or plant part which is free or substantially free of hemipteran insects.

A plant protection composition is a composition comprising an plant protection agent as described.

Suitable plants or parts thereof which may be protected by the agents of the present invention include crops and plants of agricultural, horticultural, or economic significance. Suitable plants may include any of the following or parts thereof:

Musa textilis, Medicago sativa, Prunus dulcis, Pimpinella anisum, Malus sylvestris, Prunus armeniaca, Areca catechu, Arracacia xanthorhiza, Maranta arundinacea, Cynara scolymus, Helianthus tuberosus, Asparagus officinalis, Persea americona, Pennisetum americanum, Vigna subterranean, Musa paradisiaca, Hordeum vulgare, Phaseolus vulgaris, Phaseolus vigna spp., Beta vulgaris, Citrus bergamia, Rubus spp., Piper nigrum, Acacia mearnsii, Vaccinium spp., Bertholletia excelsa, Artocarpus altilis, Viciafaba, Brassica oleracea botrytis, Sorghum bicolor, Brassica oleracea gemmifera, Fagopyrum esculentum, Brassica oleracea capitate, Brassica rapa, Brassica spp., Theobroma cacao, Cucumis melo, Carum carvi, Elettaria cardamomum, Cynara cardunculus, Ceratonia siliqua, Daucus carota, Anacardium occidentale, Manihot esculenta, Ricinus communis, Brassica oleracea botrytis, Apium graveolens, Sechium edule, Prunus spp., Castanea sativa, Cicer arietinum, Cichorium intybus, Cichorium intybus, Capsicum spp., Cinnamomum verum, Cymbopogon nardus, Citrus medica, Citrus veticulata, Trifolium spp., Syzygium aromaticum, Cocos nucifera, Colocasia spp.; Xanthosoma spp., Coffee spp., Cola spp., Brassica napus, Zea mays, Valerianella locusta, Gossypium spp., Vigna unguiculate, Vaccinium spp., Lepidium sativum, Cucumis sativus, Ribes spp., Annona reticulata, Colocasia esculenta, Phoenix dactylifera, Moringa oleifera, Phaseolus spp., Allium sativum, Allium cepa, Pisum sativum, Triticum durum, Xanthosoma spp.; Colocasia spp., Solanum melongena, Cichorium endivia, Lygeum spartum, Foeniculum vulgare, Trigonella foenumgraecum, Ficus carica, Corylus avellane, Furcraea macrophylla, Linum usitatissimum, Phormium tenax, Pelargonium spp.; Geranium spp., Zingiber officinalis, Langenaria spp; Cucurbita spp., Cicer arietinum, Citrus paradise, Vitis vinifera, Lygeum spartum, Dactylis glomerata, Arachis hypogaea, Psidium guajava, Corylus avellane, Cannabis sativa, Crotalaria juncea, Agave fourcroydes, Lawsonia inermis, Humulus lupulus, Armoracia Rusticana, Indigofera tinctorial, Jasminum spp., Corchorus spp., Brassica oleracea acephala, Ceiba pentandra, Hibiscus cannabinus, Brassica oleracea gongylodes, Lavandula spp., Allium ampeloprasum, Citrus limon, Cymbopogon citratus, Lens culinaris, Lespendeza spp., Lactuca sativa, Glycyrrhiza glabra, Citrus aurantifolia, Citrus limetta, Linum usitatissimum, Litchi chinensis, Eriobotrya japonica, Lupinus spp., Macadamia spp., Myristica fragrans, Agave atrovirens, Citrus reticulata, Mangifera indica, Manihot esculenta, Secale cereal, Mespilus germanica, Cucumis melo, Penicum miliaceum, Eleusine coracana, Setaria italica, Echinochloa crusgalli, Eleusine coracana; Mentha spp., Morus spp., Morus alba, Agaricus spp.; Pleurotus spp. Volvariella, Brassica nigra; Sinapis alba, Prunus persica, Phormium tenax, Guizotia abyssinica, Myristica fragrans, Avena spp., Elaeis guineensis, Abelmoschus esculentus, Olea europea, Papaver somnferum, Citrus sinensis, Citrus aurantium, Dactylis glomerate, Metroxylon spp., Borassus flabellfer, Carica papaya, Pastinaca sativa, Pyrus communis, Pisum sativum, Carya illinoensis, Capsicum annuum, Diospyros kaki; Diospyros virginiana, Cajanus cajan, Ananas comosus, Pistacia spp., Prunus domestica, Punica granatum, Citrus grandis, Solamum tuberosum, Ipomoea batatas, Cucurbita spp., Chrysanthemum cineraraiefolium, Aspidosperma spp., Cydonia oblonga, Cinchona spp., Chenopodium quinoa, Raphanus sativus (including Cochlearia armoracia), Boehmeria nivea, Agrostis spp., Boehmeria nivea, Rheum spp., Oryza sativa; Oryza glaberrima, Rose spp., Hevea brasiliensis, Secale cereal, Lolium spp., Carthamus tinctorius, Metroxylon spp., Onobrychis viciifolia, Valerianella locusta, Tragopogon porrifolius, Achras sapota, Citrus reticulata, Brassica ileracea capitate, Scorzonera hispanica, Sesamum indicum, Butyrospermum paradoxum, Agave sislana, Citrus aurantifolia, Glycine max, Triticum spelta, Spinacia oleracea, Secale cereal, Cucurbita spp., Fragaria spp., Sorghum bicolor Sudanense, Saccharum officinarum, Helianthus annuus, Crotalaria juncea, Citrus limetta, Iopmoea batatas, Citrus reticulata, Xanthosoma sagittifolium, Manihot esculenta, Colocasia esculenta, Camellia sinensis, Eragrostis abyssinica, Phleum pratense, Nicotiana tabacum, Lycopersicum esculentum, Lotus spp., Aleurites spp., Brassica rapa, Urena lobate, Vanilla planfolia, Vicia sativa, Juglans spp., Citrullus lanatus, Acacia mearnsii, Triticum spp., Hordeum spp., Dioscorea spp., and Ilex paraguariensis.

Suitably, the plant or part thereof which may be protected by the agents of the present invention is selected from a plant which suffers from hemipteran or dipteran insect infestations, or which attracts hemipteran or dipteran insects. Suitably, the plant or part thereof which suffers from hemipteran or dipteran insect infestations, or which attracts hemipteran or dipteran insects is any of those listed above.

In one embodiment, the plant is selected from a plant which suffers from or attracts hemipteran insect infestations, for example: cereal crops such as wheat (Triticum spp.), oats (Avena spp.), rye (Secale spp.), barley (Hordeum spp.), rice (Oryza spp.) and corn (Zea spp.); fruit and vegetable crops including apples (Malus spp.); pears (Pyrus spp.); strawberry (Fragaria spp.), blueberry (Vaccinum spp.), blackberry (Rubus spp.), raspberry (Rubus spp.), citrus (Citrus spp.), olive (Olea spp.), durian (Durio spp.), longan (Dimocarpus spp.), litchi (L. chinensis), persimmon (Diospyros spp.); beans and peas (including but not limited to Phaseolus, Vigna, Pisum, Lens, Glycine, Cicer, Cajanus, Arachis spp.), sugar beet (Beta vulgaris), sugar cane (Saccharum spp.), lettuce (Lactuca spp.), brassicas (Brassica spp.) including oil seed rape, alliums (Allium spp.), tomato (Solanum spp.), pepper (Capsicum spp.), asparagus (A. officinalis), melon, squash, pumpkins (Cucumis spp.), and tubers (potato) (Solanum spp.), or a part thereof.

In one embodiment, the plant is selected from a plant which suffers from or attracts aphid insect infestations, suitably M. persicae insect infestations, including Solanaceae, Cruciferae, and Leguminosae for example: cereal crops such as wheat (Triticum spp. including winter wheat Triticum aestivum L); fruit and vegetable crops including peach (Prunus spp.), strawberry (Fragaria spp.), blueberry (Vaccinum spp.), blackberry (Rubus spp.), raspberry (Rubus spp.), brassicas (Brassica spp.) such as oil seed rape, lettuce (Lactuca spp.), tomato (Solanum spp.), pepper (Capsicum spp.), beans and peas (including but not limited to Vigna, Pisum spp.), melon, squash, pumpkins (Cucumis spp.), citrus (Citrus spp.), and tubers (potato) (Solanum spp.), or a part thereof In one embodiment, the plant is a vegetable crop, suitably a brassica spp.

In one embodiment, the plant is selected from a plant which suffers from or attracts dipteran insect infestations, for example: cereals (Triticum spp.); oats (Avena spp.);, rye (Secale spp.); barley (Hordeum spp,) rice (Oryza spp.) and corn (Zea spp.); beans and peas (including but not limited to Phaseolus, Vigna, Pisum, Lens, Glycine, Cicer, Cajanus, Arachis spp.); fruit crops including apples (Malus spp.), pears (Pyrus spp.), strawberry (Fragaria spp.), blueberry (Vaccinum spp.), blackberry (Rubus spp.), raspberry (Rubus spp.), cherry, plum, apricot, peach, nectarine (Prunus spp.), blackcurrant, redcurrant, whitecurrant, gooseberry (Ribes spp.), kiwi fruit (Actinidia spp.), papaya (Carica spp.), avocado (Persea spp.), mango (Mangifera indica L), longan (Dimocarpus spp.), litchi (L. chinensis), grapes (Vitis spp.), fig (Ficus spp.), passionfruit (Passiflora spp.), Asian pears (Pyrus spp.), citrus (Citrus spp.), and olive (Olea spp.); vegetable crops including alliums (Allium spp.), aubergine, tomato (Solanum spp.) and peppers (Capsicum spp.), lettuce (Lactuca spp.), brassicas (Brassica spp.) and courgette, melon, squash, pumpkins (Cucumis spp.); Apiaceae root crops including carrot (Daucus spp.), parsnip (Pastinaca spp.), or a part thereof.

In one embodiment, the plant is selected from a plant which suffers from or attracts fly insect infestations, suitably D. suzukii insect infestations, for example: fruit crops including strawberry (Fragaria spp.); blueberry (Vaccinum spp.); blackberry (Rubus spp.); raspberry (Rubus spp.); cherry, plum, apricot, peach, nectarine (Prunus spp.); blackcurrant, redcurrant, whitecurrant, gooseberry (Ribes spp.); fig (Ficus spp.); citrus (Citrus spp.), Asian pears (Pyrus spp.); or a part thereof.

### Hemipteran Insects

The compounds and compositions of the invention may have activity against insects of the Order Hemiptera, which comprises groups including aphids, planthoppers, leafhoppers, stink bugs, shield bugs and cicadas.

Hemipterans are defined by distinctive mouthparts in the form of a “beak”, comprising modified mandibles and maxillae which form a “stylet”, sheathed within a modified labium.

Many insects within these groups have endogenous neuropeptides with sequence homology to the peptides described herein, suggesting that these analogues may have activity against those insects.

The insects may belong to the sub-order Sternorrhyncha, e.g. to the super-family of Aphidoidea (aphid superfamily), Aleyrodoidea (whiteflies), Coccoidea (scale insects), Phylloxeroidea (including Phylloxeridae or “phylloxerans”, and Adelgidae or woolly conifer aphids) or Psylloidea (jumping plant lice etc.).

Thus, the insects may be aphids, i.e. members of the aphid superfamily (Aphidoidea).

Aphids (Hemiptera: Aphididae) are one of the most significant groups of agricultural pests³⁸ and are vectors in the transmission of approximately 50% of all insect transmitted plant viruses.³⁹ Within that superfamily, the aphids may be part of the family Aphididae, which contains sub-families Aiceoninae, Anoeciinae, Aphidinae, Baltichaitophorinae, Calaphidinae, Chaitophorinae, Drepanosiphinae, Eriosomatinae, Greenideinae, Hormaphidinae, Israelaphidinae, Lachninae, Lizeriinae, Macropodaphidinae, Mindarinae, Neophyllaphidinae, Phloeomyzinae, Phyllaphidinae, Pterastheniinae, Saltusaphidinae, Spicaphidinae, Taiwanaphidinae, Tamaliinae and Thelaxinae.

The aphids may, for example, be of the genus Acyrthosiphon (e.g. Acyrthosiphon pisum), Aphis (e.g. Aphis gossypii, Aphis glycines), Diuraphis (e.g. Diuraphis noxia) Macrosiphum (e.g. Macrosiphum rosae, Macrosiphum euphorbiae), Myzus (e.g. Myzus persicae), or Sitobion (e.g. Sitobion avenae).

Myzus persicae (peach potato aphid) is the most economically important aphid crop pest worldwide,⁴⁰ with a global distribution and host range encompassing more than 400 species in 40 different plant families.⁴¹ For example, it is a major pest of agricultural crops including fruit and potatoes, and act as a vector for viruses.

Macrosiphum rosae, (rose aphid) is an important horticultural pest, especially of cultivated species of Rosa, and is a vector in the transmission of 12 plant viruses including the strawberry mild yellow edge virus.⁴¹

Aphis gossypii (cotton or melon aphid) is a pest of Curcibitae and cotton.

Other than aphids, the insects may, for example, be of the Adelgidae family, e.g. of the genus Adelges (e.g. Adelges tsugae).

The insects may be of the Aleyrodidae family, e.g. of the genus Bemisia (e.g. Bemisia tabaci) or Trialeurodes (e.g. Trialeurodes vaporariorum).

The insects may be of the Psylloidea family, e.g. of the genus Pachypsylla (e.g. Pachypsylla venusta).

As examples of hemipteran insects outside the sub-order Sternorryncha, the insects may be of the Cimicidae family, e.g. of the genus Cimex (bed bugs), e.g. Cimex lectularius.

The insects may be of the Cicadellidae family, e.g. of the genus Cuerna (e.g. Cuerna arida), Graminella (e.g. Graminella nigrifrons) or Homalodisca (e.g. Homalodisca vitripennis).

The insects may be part of the Delphacidae family, e.g. of the genus Nilaparvata (e.g. Nilaparvata lugens) or Sogatella (e.g. Sogatella furcifera). For example, Nilaparvata lugens (brown planthopper) is a pest of rice crops, especially in Asia.

The insects may be of the Liviidae family, e.g. of the genus Diaphorina (e.g. Diaphorina citri).

The insects may be part of the Miridae family, e.g. of the genus Pseudatomoscelis (e.g. Pseudatomoscelis seriatus), Lygus (e.g. Lygus hesperus) or Tupiocoris (e.g. Tupiocoris notatus). For example, Pseudatomoscelis seriatus (cotton fleahopper) is a pest of cotton.

The insects may be of the Pentatomidae family, e.g. of the genus Acrosternum (e.g. Acrosternum hilare), Banasa (e.g. Banasa dimiata), Euschistus (e.g. Euschistus servus, Euschistus heroes), Halyomorpha (e.g. Halyomorpha halys), Murgantia (e.g. Murgantia histrionica), Nezara (e.g. Nezara viridula), Plautia (e.g. Plautia stali), or Podisus (e.g. Podisus maculiventris). For example, Acrosternum hilare (green stink bug) is a significant pest of cotton. Euschistus servus (brown stink bug) is a pest of many agricultural crops including seeds, grains, nuts and fruits, especially in the southern USA. Nezara viridula is a pest of grain and soybean crops, especially in Brazil.

The insects may be of the Pyrrhocoridae family, e.g. of the genus Pyrrhocoris (e.g. Pyrrhocoris apterus).

The insects may be of the Reduviidae family, e.g. of the genus Rhodnius (e.g. Rhodnius prolixus), or Triatoma (e.g. Triatoma infestans). Rhodnius prolixus is a vector of human disease (Chagas disease).

The insects may be of the Triozidae family, e.g. of the genus Acanthocasuarina (e.g. Acanthocasuarina muellerianae).

In one embodiment, the insect may be selected from the following species: H. halys, E. heroes, A. hilare, A. gossypii, E. servus, M persicae, N. viridula, N. lugens, P. seriatus, and R. prolixus.

In one embodiment, the insect is of the species M. persicae.

### Dipteran Insects

The compounds and compositions of the invention may have activity against insects of the Order Diptera,

In particular, they may have activity against insects of the family Drosophilidae, such as fruit flies, including those of genus Drosophila, such as Drosophila suzukii. They may also have activity against insects of the family Tephritidae, including those of the genera Anastrepha (Anastrepha spp.); Bactrocera (Bactrocera spp.); Ceratitis (Ceratitis spp.); Dacus (Dacus spp.); Rhagoletis (Rhagoletis spp.); Tephritis (Tephritis spp.).

The families Drosophilidae and Tephritidae together are commonly referred to as fruit flies.

The compounds may also have activity against other important dipteran pests, such as flies of the family Chloropidae (chloropid flies) and those of the genera:
Phytomyza (e.g. Phytomyza angelicastri);
Melani (e.g. Melani agromyza);
Antherigona (e.g. Antherigona spp.);
Delia (e.g. Delia radicum);
Contarinia (e.g. Contarinia sorghicola);

In one embodiment, the insect is of the species Drosophila suzukii.

(For more detail on these, and other examples, see Developing the Arsenal Against Pest and Vector Dipterans: Inputs of Transgenic and Paratransgenic Biotechnologies, Ogaugwu and Durvasula, IntechOpen, 2017: DOI:10.5772 66440

### Preferred Embodiments of the Invention

The present invention describes the use of a compound as described herein as an insect control agent, specifically in methods of increasing hemipteran and/or dipteran insect mortality, or a method of inhibiting infestation of a plant by hemipteran and/or dipteran insects.

Suitably, the compound may be for use as an insect control agent wherein the insect is of the order dipteran, and wherein the compound is selected from: AH56, AH188, AH382, AH383, AH270/AHPEG270. Suitably, the compound may be for use as an insect control agent wherein the insect is of the genus Drosophila, and wherein the compound is selected from: AH56, AH188, AH382, AH383, AH270, AHPEG270. Suitably, the compound may be for use as an insect control agent wherein the insect is Drosophila suzukii, and wherein the compound is selected from: AH56, AH188, AH382, AH383, AH270/AHPEG270.

In one embodiment, there is provided a method of increasing dipteran insect mortality, comprising contacting a dipteran insect or dipteran insect population with a compound selected from AH56, AH188, AH382, AH383, AH270/AHPEG270.

In one embodiment, there is provided a method of inhibiting infestation of a plant by dipteran insects comprising contacting the plant with a compound selected from AH56, AH188, AH382, AH383, AH270/AHPEG270.

In some embodiments, the compound for use against insects of the order diptera is selected from: AH188, AH382, AH383, AH270/AHPEG270. In some embodiments, the compound for use against insects of the order diptera is AH382.

In one particular embodiment, there is provided a method of increasing Drosophila suzukii mortality, comprising contacting a Drosophila suzukii insect or insect population with compound AH382.

In one particular embodiment, there is provided a method of inhibiting infestation of a plant by Drosophila suzukii comprising contacting the plant with compound AH382.

Suitably contacting may comprise feeding or spraying, for example. Suitably feeding may be encouraged via bait attractants, which may be comprised in a composition of the invention, as explained below.

Suitably, the compound may be for use as an insect control agent wherein the insect is of the order hemipteran, and wherein the compound is selected from: AH270/AHPEG270, AH257, AH259, AH383, AH387. Suitably, the compound may be for use as an insect control agent wherein the insect is of the genus Myzus, and wherein the compound is selected from: AH270/AHPEG270, AH259, AH383, AH387. Suitably, the compound may be for use as an insect control agent wherein the insect is Myzus persicae, and wherein the compound is selected from: AH270/AHPEG270, AH257, AH259, AH383, AH387.

In one embodiment, there is provided a method of increasing hemipteran insect mortality, comprising contacting a hemipteran insect or hemipteran insect population with a compound selected from AH270/AHPEG270, AH257, AH259, AH383, AH387.

In one embodiment, there is provided a method of inhibiting infestation of a plant by hemipteran insects comprising contacting the plant with a compound selected from AH270/AHPEG270, AH257, AH259, AH383, AH387.

In some embodiments, the compound for use against insects of the order hemiptera is selected from: AH383, AH387, AH257, AH259. In some embodiments, the compound for use against insects of the order hemiptera is AH387.

In one particular embodiment, there is provided a method of increasing Myzus persicae mortality, comprising contacting a Myzus persicae insect or insect population with a compound selected from: AH383, AH387, AH257, AH259.

In one embodiment, there is provided a method of inhibiting infestation of a plant by Myzus persicae comprising contacting the plant with a compound selected from: AH383, AH387, AH257, AH259.

In one particular embodiment, there is provided a method of increasing Myzus persicae mortality, comprising contacting a Myzus persicae insect or insect population with compound AH387.

In one particular embodiment, there is provided a method of inhibiting infestation of a plant by Myzus persicae comprising contacting the plant with compound AH387.

Suitably contacting may comprise feeding or spraying, for example. In some embodiments, when the contacting is by feeding, the compound may be selected from: AH383 or AH387. In some embodiments, when the contacting is by spraying, the compound may be selected from AH257, or AH259.

Suitably the compound may be contacted with the insect or insect population, or plant or plant part, at any suitable concentration which is effective. Suitably the concentration of the compound is between 10⁻³ to 10⁻⁹ M, suitably between 10⁻⁴ to 10⁻⁶ M, suitably between 10⁻⁴ to 10⁻⁵M.

### Pollinator Species

The compounds and compositions of the invention may be substantially non-toxic to beneficial insect species. These important pollinator species, such as insects of the superfamily Apoidea, including bees, such as the Apidae, e.g. those of the genus Bombus, such as Bombus terrestris.

By substantially non-toxic, it is meant that the compounds and compositions of the invention do not cause death of the pollinator species, suitably that they do not cause premature death of the pollinator species. It is also meant that the compounds and compositions of the invention do not cause any detrimental side effects to the pollinator species, for example they do not have a negative effect on feeding behaviour, or ability to move.

### Compositions

Compositions of the invention, or for use in accordance with the invention, typically comprise a compound as described in combination with one or more ancillary component such as solvents, carriers, diluents, adjuvants, preservatives, dispersants, emulsifying agents, or synergists.

The compound content of the composition can vary within wide limits. The compound concentration of the composition can be from 0.0000001 to 95% by weight of the compound, preferably between 0.0001 and 1% by weight.

The compositions of the invention, or for use in accordance with the invention, may comprise more than one compound of the invention in combination. Therefore the compositions of the invention may comprise a first compound of the invention and a second compound of the invention. Suitably the first and second compound may be any of those described herein, and may be present in the composition in any relative proportion. In one embodiment, the first compound may be a CAPA1 analogue and the second compound may be a CAPA2 analogue.

The composition may be an aqueous composition, e.g. a saline composition. The aqueous composition may contain one or more buffers, such as a phosphate buffer (e.g. phosphate buffered saline) or a Tris buffer. Alternatively the composition may be an oil dispersion or an emulsion, e.g. an oil and water emulsion. Alternatively the composition may be a suspension, powder, foam, paste, granule, aerosol, impregnated natural and synthetic substance, or encapsulated in polymeric substance for example. A suitable form of the composition may be chosen for the intended use having regard to the target insect, and to its habitat.

Adjuvants may enhance product performance, for example, by increasing the efficiency of the delivery of active ingredients, reducing the level of active ingredient required, or extending the spectrum of effectiveness.

Different types of adjuvants offer various benefits and advantages, which are achieved by modulating properties such as spray formation, spray retention, wetting, deposit formation or uptake.

Adjuvants modulating spray formation may influence spray quality by reducing spray drift and wastage, allowing more of the product to reach the target. This can reduced use rates, leading to a better environmental profile and a potentially more cost effective solution. Such adjuvants include non-ionic surfactants and emulsifier blends.

Adjuvants modulating spray retention may dissipate the kinetic energy of the droplet during impact, meaning the likelihood of bounce or run-off is reduced. Such adjuvants include alkyl polyglucosides, alkoxylated alcohols, and polyoxyethylene monobranched alcohols (e.g. polyoxyethylene (8) monobranched alcohol).

Adjuvants modulating wetting properties (i.e. wetting agents) may reduce surface tension and contact angle, leading to enhanced coverage. Such adjuvants include polyoxyethylene sorbitan monolaurate (e.g. polyoxyethylene (8) sorbitan monolaurate), surfactant blends, and alkyl polyglucosides.

Adjuvants modulating deposit formation may influence evaporation of water from the droplet and thus provide a more homogeneous distribution. Such adjuvants include alkoxylated polyol esters, polyoxyethylene sorbitan monolaurate (e.g. polyoxyethylene (12) sorbitan monolaurate), and alkyl polyglucoside.

Adjuvants modulating uptake can improve penetration and uptake of active ingredients. e.g. through the insect cuticle, resulting in increased bioavailability. Such adjuvants include alkoxylated polyol esters and polyoxyethylene sorbitan monolaurate (e.g. polyoxyethylene (12) sorbitan monolaurate and polyoxyethylene (16) sorbitan monolaurate).

Dispersants may be aqueous or non-aqueous. An oil dispersion (OD) formulation typically comprises a solid active ingredient dispersed in oil. The oil can vary from paraffinic to aromatic solvent types and vegetable oil or methylated seed oils. Typically the active ingredient is uniformly suspended in the oil phase. Although primarily used for water sensitive active ingredients, OD formulations have extended to other active ingredients due to their better spray retention, spreading, foliar uptake, and penetration enhancement (e.g. across the insect cuticle) as the carrier oil often acts as an adjuvant.

Oils suitable for use in OD dispersions include linseed, rapeseed and soyabean oils.

Aqueous dispersants may be used, for example, to improve stability in the spray tank after dilution in water, and may include modified styrene acrylic polymers, and polymeric amphoteric dispersants and adjuvants.

An emulsifier may be employed to emulsify a continuous oil phase into water when an OD formulation is diluted prior to being sprayed. The emulsifier may be selected based upon its ability to spontaneously form the emulsion. Their performance is primarily dictated by the nature of the surfactant and their collective effect on how they arrange themselves at the oil/water interface. Examples include polyoxyethylene sorbitol hexaoleate (e.g. polyoxyethylene (40) sorbitol hexaoleate), emulsifier blends, and calcium alkylaryl sulphonate.

The compound may further comprise an adhesive or a dye.

The compound may be provided in the form of a concentrate, for dilution prior to application. Alternatively the compound may be provided in a solid form to be suspended or dissolved prior to formulation.

The composition may be a bait composition for ingestion by the target insect. A bait composition may comprise one or more phagostimulants, i.e. a substance which will entice the insect to ingest the compound. Phagostimulants may include artificial sweeteners, amino acids, other peptides or proteins and carbohydrates (e.g. glucose, fructose, sucrose, maltose) etc.. Examples include honey, syrups and aqueous solutions of sucrose.

Commercially available base formulations may also be suitable for use in formulating the compounds described in this specification, such as Armid® FMPC (Akzo Nobel).

The composition may comprise one or more synergists, i.e. compounds which increase the efficacy of insecticides against their targets, often by inhibiting an insect's ability to metabolise the active agent. Common synergists include piperonyl butoxide and MGK-264 (n-octyl bicycloheptane dicarboximide), or peptidase inhibitors.

The composition may further comprise one or more additional active insecticides, such as (but not limited to) pyrethrins or pyrethroids, or other peptide analogues. The insecticides may also include, for example, phosphates, carbamates, carboxylates, chlorinated hydrocarbons, phenylureas and substances produced by microorganisms.

The choice of ancillary or additional insecticides will typically depend on the particular target species. The composition may further comprise one or more additional, attractants, sterilizing agents, acaricides, nematicides, fungicides, growth-regulating substances or herbicides.

### EXAMPLES

### Compounds

The following peptide compounds were synthesised:

Note: ‘H’ indicates N terminal hydrogen, ‘NH₂’ indicates C terminal amidation, synthetic amino acids are defined using the nomenclature above, as is ‘guanidyl’, ‘Me’ indicates methylation as defined above

TABLE 1

| CAPA1 variants | Structure | SEQ ID NO: |
|---|---|---|
| AH188 | H-LYAFARV-NH₂ | 20 |
| AH380 | H-LYAFAR-(Me)V-NH₂ | 21 |
| AH382 | H-LY-(Me)A-FARV-NH₂ | 22 |
| AH383 | H-(Me)L-YAFARV-NH₂ | 24 |
| AH387 | H-LY-Aib-FARV-NH₂ | 25 |
| CAPA2 variants | Structure | SEQ ID NO: |
| AH56 | H-Ahx-LVAFPRV-NH₂ | 5 |
| AH257 | Guanidyl-LVAFPR-(Me)V-NH₂ | 6 |
| AH259 | Guanidyl-L-(Me)V-AFPRV-NH₂ | 7 |
| AH283 | H-Ahx-L-(Me)V-AFPRV-NH₂ | 8 |
| AH270 | H-Ahx-LV-(Me)A-FPR-(Me)V-NH₂ | 10 |

The following control peptides were also synthesised, having the sequences of native Capa1 and Capa2 from D. melanogaster:

TABLE 2

| Control peptides | Structure | SEQ ID NO: |
|---|---|---|
| CAPA1 | H-GANMGLYAFPRV-NH₂ | 31 |
| CAPA2 | H-ASGLVAFPRV-NH₂ | 32 |

Peptides were based on rational design from bioinformatics analysis of native Capa peptide sequences in the common pests M. persicae and D. suzukii.

M. persicae Capa peptide sequences were interrogated from our DiNER database of insect neuropeptides (Yeoh et al., Insect Biochem and Mol Biol, 2018), Table 3 below.

| | | Species | Amino Acid | |
|---|---|---|---|---|
| Isoform | Neuropeptide | Name | Sequence | |
| Myzpe- | CAPA | Myzus | ESVAGLIPFPRVamide | SEQ ID NO: 33 |
| CAPA-1 | | persicae | | |
| Myzpe- | CAPA | Myzus | EGLIPFPRIamide | SEQ ID NO: 34 |
| CAPA-2 | | persicae | | |
| Myzpe- | CAPA | Myzus | SDSKNTALWFGPRLamine | SEQ ID NO: 35 |
| CAPA-3 | | persicae | | |

M. persicae has 3 capa peptides: Myzpe capa1 with the FPRV motif, capa-2 with a FPRI motif, and capa-3 with a PRL motif. As FPRV is critical for binding to the cognate Capa receptor, peptides designed with the FPRV motif were envisaged to be effective against M. persicae (Myzpe capa1).

D. suzukii Capa peptide sequences were also interrogated from our DiNER database of insect neuropeptides (Yeoh et al., Insect Biochem and Mol Biol, 2018). Sequences were retrieved for the important beneficial pollinator species Apis mellifera (Apime, honeybee) and B. terrestris (Bomte, pollinator bumblebee); and the D. suzukii (Drosu, SWD) pest species, Table 4 below.

| Isoform | Neuropeptide | Species Name | Amino Acid Sequence | |
|---|---|---|---|---|
| Apime- | CAPA | Apis mellifera | AFGLLTYPRIamide | SEQ ID NO: 36 |
| CAPA-2 | | | | |
| Bomte- | CAPA | Bombus | ASGLMAYPRVamide | SEQ ID NO: 37 |
| CAPA-1 | | terrestris | | |
| Drosu- | CAPA | Drosophila | GANMGLYAFPRVamine | SEQ ID NO: 38 |
| CAPA-1 | | suzukii | | |
| Drosu- | CAPA | Drosophila | ASGLVAFPRVamide | SEQ ID NO: 39 |
| CAPA-2 | | suzukii | | |

Apime has 1 capa peptide, with a YPRI motif Bomte has 1 capa peptide, with a YPRV motif The Drosu genome encodes 2 capa peptides, identical to capa 1 and 2 in the genetic model insect, D. melanogaster (Kean, Am. J. Physiol., 2002). Note the FPRV motif, required for binding to the cognate D. melanogaster capa receptor (Kean, Am. J. Physiol., 2002; Terhzaz et al., PLoS One, 2012; Halberg et al., Nature Comms, 2015, Terhzaz et al., Pest Mgt Science, 2017).

FPRV is critical for binding to the cognate capa receptor—as such, peptides designed with the FPRV motif were envisaged to be specific for M. persicae and D. suzukii but have a low probability to affect honeybees or bumblebees.

In order to verify the hot spot binding residues of the Capa peptide, an alanine scan was performed and a truncation series of the Drosophila melanogaster Capa-1 peptide designed and synthesised. The replacement of Phe5 by alanine markedly reduced the observed Calcium response, which demonstrated the key requirement of this residue for binding to CapaR. We established the required minimal heptameric pharmacophore of the Capa peptide (LYAFPRV SEQ ID NO: 40).

This core sequence was then used to design biostable and bioactive capa peptides with various modifications such as addition of guanidine, methylation and substitution with artificial amino acid residues, to increase resistance to enzymatic degradation. A series of N-terminal moieties was introduced ranging from lipophilic, aliphatic and aromatic, to aid cuticle permeability.

In summary, the active core of capa peptides which are used for receptor activation was identified; identification of necessary amino acids outside the core sequence which also resulted in antagonists of capaR was accomplished; and N-terminal modifications for agonistic receptor activity were added. First to Fourth generations of peptides were produced with varying structures and tested for CAPA receptor agonist activity, and screened for lethality in vivo against D. suzukii and M. persicae as per methods in example 2 and example 8. Some of these peptides are shown in the table below. The top performing peptides were selected and gave rise to the list of CAPA1 and CAPA2 peptides provided above in Table 1 which were then further tested in the following applied examples.

TABLE 5
Peptide screening

| | | | D. | D. suzukii | M. |
|---|---|---|---|---|---|
| AH | | | suzukii | (Mean% | persicae |
| peptide_ | SEQ | | (Sig | Lethality ± | (% |
| ID | ID NO | Peptide_sequence | Diff Grp) | SD) | Lethality) |
| Blank | | | | 20.33 ± | |
| Control | | | | 12.38 | |
| AH283* | 8 | H-Ahx-L-(Me)V-AFPRV- | A | 42.63 ± | 40% |
| | | NH₂ | | 23.92 | |
| AH259* | 7 | Guanidyl-L-(Me)V-AFPRV- | A | 40.63 ± | |
| | | NH₂ | | 17.93 | |
| AH258 | 11 | Guanidyl-LV-(Me)A-FPRV- | A | 47.75 ± | |
| | | NH₂ | | 21.93 | |
| AH257* | 6 | Guanidyl-LVAFPR-(Me)V- | A | 45.57 ± | 25% |
| | | NH₂ | | 18.32 | |
| AH188* | 20 | H-LYAFARV-NH₂ | ABC | 70.50 ± | 22% |
| | | | | 23.01 | |
| AH62 | 28 | Palmitoyl-LYAFPRV-NH₂ | — | — | 29% |
| AH59 | 12 | Palmitoyl-LVAFPRV-NH₂ | A | 44.13 ± | |
| | | | | 19.29 | |
| AH56* | 5 | H-Ahx-LVAFPRV-NH₂ | AB | 50.63 ± | 30% |
| | | | | 19.28 | |
| AH55 | 13 | 4-Benzoyl benzoic- | AB | 54.50 ± | |
| | | LVAFPRV-NH₂ | | 16.45 | |
| AH51 | 29 | H-Nle-LYAFPRV-NH₂ | A | 38.63 ± | — |
| | | | | 16.49 | |
| AH49 | 26 | 4-Benzoyl benzoic- | A | 41.13 ± | |
| | | LYAFPRV-NH₂ | | 17.01 | |
| AH33 | 14 | Ac-LVAFPRV-NH₂ | A | 43.50 ± | |
| | | | | 14.50 | |
| AH32 | 27 | Ac-LYAFPRV-NH₂ | A | 41.50 ± | |
| | | | | 21.49 | |

*indicates peptides taken forward for further testing
‘Ac’ means acetyl
A indicates a significant difference in survival compared with control (blank).
B indicates a significant difference in survival compared with scrambled capa1.
C indicates a significant difference in survival compared with scrambled capa2.

### Peptide Synthesis, Purification and Characterization

Peptides were synthesized by solid phase peptide synthesis (SPPS) using an Fmoc /tBu approach on Fmoc-Rink Amide AM resin. Peptides were assembled on a Biotage Syro II, Biotage Initiator Alstra or a PTI Tribute synthesizer using Fmoc-amino acids and HCTU/DIPEA mediated coupling reactions. Fmoc SPPS utilized a capping step after coupling, to ensure acylation of unreacted free amines.

Peptide purification was carried out via RP-TIPLC on an Agilent 1260 Infinity Preparative RP-TIPLC system. Peptides were purified using a Dr. Maisch, Reprosil Gold, C18, 250 mm×20 mm, 10 μm column. Peptide purity was subsequently assessed by analytical RP-HPLC on a Shimadzu analytical HPLC system, using a Dr. Maisch, Reprosil Gold, C18, 250 mm×4.6 mm, 5 μm column.

Compound characterization was performed using both low and high resolution ESI-MS (peptides). Components were separated via RP-HPLC on a Thermo Scientific Dionex Ultimate 3000 system and subsequently analyzed for mass to charge (m/z) ratio on a Thermo Scientific LCQ-FLEET Electrospray Ionization (ESI) system in positive ion mode. For high resolution MS, a Bruker MicroTOF Q was used. Where appropriate, ¹H 1D, ¹H ¹³C HSQC and ¹³C 1D spectra were recorded on a Bruker 400 MHz Ultrashield spectrometer in Deuterated solvents. ¹H 1D NMR spectra were assigned from chemical shift values, combined with HSQC coupling patterns.

N,N′-Bis-dimethyl guanidine groups were introduced by reacting resin bound peptide amines (0.025 mmol) with 0.5 M HCTU/DMF (eq. =4, n=0.1 mmol, v=0.2 ml) and 1.0 M DIPEA/DMF (eq. =8, n=0.2 mmol, v=0.2 ml) in DMF (v=0.2 ml) with agitation at ambient temperature for 30 min. Once reagents had been drained, the peptide resin was then washed with DMF (5×0.6 ml×45 s).

All peptides were purified to >90% as determined by two gradients of analytical HPLC.

### Aphid Rearing

Stock cultures of anholocyclic M. persicae were established using aphids supplied by the Smagghe laboratory, Ghent University, Belgium. Cultures were reared under a 12:12 h LD photocycle at 22° C. on Chinese cabbage (Brassica rapa var. Wong Bok) contained within a BugDorm fine mesh cage (44545F) (45 cm×45 cm×45 cm). A fresh supply of Chinese cabbage of approximately 4 weeks from sowing was supplied to the cages on a once-weekly basis to maintain the aphid cultures.

### Example 1

Feeding of Aphids with Peptides in Artificial Diet

A standard artificial diet for M. persicae was produced as described in Van Emden (2009) and provided the basal diet to which peptides were added for screening purposes. Peptides were diluted individually in the artificial diet to a pre-determined concentration.

Feeding apparatus were constructed using a set-up developed by Sadeghi et al (2009). For this, a piece of Parafilm was stretched over a Plexiglas ring (h=4 cm, Ø=3 cm) and 100 μl of the artificial diet containing the desired neuropeptide analogue was pipette onto the Parafilm membrane. A second piece of Parafilm, stretched to 4 times the original thickness, was stretched over the original layer, sandwiching the artificial diet between two layers of Parafilm. A strip of Parafilm was wrapped around the circumference of the Plexiglas ring, sealing in the diet. A plastic ring (h=1.2 cm, Ø=3.4 cm) was subsequently placed over the Parafilm layer, creating a walled chamber in which to house test aphids in contact with the Parafilm layer containing the artificial diet. Finally, a small Petri dish (h=1 cm, Ø=3.6 cm), modified for ventilation with net cloth, was placed on top of each feeding apparatus to prevent aphid escape.

To obtain aphids for use in experiments, reproducing anholocyclic adults were placed on individual excised leaves of Chinese cabbage at densities of 5 adults per leaf and allowed to reproduce for 24 h. The stem of each excised leaf was held within a 0.5 mL Eppendorf tube containing water via a punctured hole in the Eppendorf lid, and placed individually within a microcage (L=4 cm, Ø=9.5 cm). Following 24h, adults were removed and resultant first instar nymphs (<1 day old and synchronised in age to within 24h) retained. Nymphs were allowed to develop on the Chinese cabbage for 5 days. On day 5, (3^{rd} instar) nymphs were transferred onto the artificial diet containing a neuropeptide analogue at densities of 1 per feeding chamber and monitored daily until death. Aphids were transferred to fresh artificial diet (containing neuropeptide analogue or water control) every 5 days.

The water control group was used to determine baseline lethality. Results for neuropeptide analogues were calculated as % lethality in the remaining population after adjustment for the baseline. These results are shown in Tables 7 and 8 below.

### Example 2

Feeding of Aphids with Peptides in Artificial Diet

The aphid feeding protocol was devised according to Sadeghi et al (2009) as explained in example 1. Feeding discs were designed and manufactured as above. A standard sucrose-based artificial diet for M. persicae (30 aphids per chamber, 3 chambers per experiment unless otherwise stated) was produced as described in example 1 (Sadeghi et al, J. Insect Science, 2009) and provided the basal diet to which peptides were added for screening purposes. Peptides were diluted individually in the artificial diet to 10⁻⁵ M. Lethality was scored after up to 120 hours (IRAC guidelines for aphid testing).

Membrane discs were also included to collect ‘honeydew’ secreted by feeding aphids and stained with ninhydrin to observe feeding patterns.

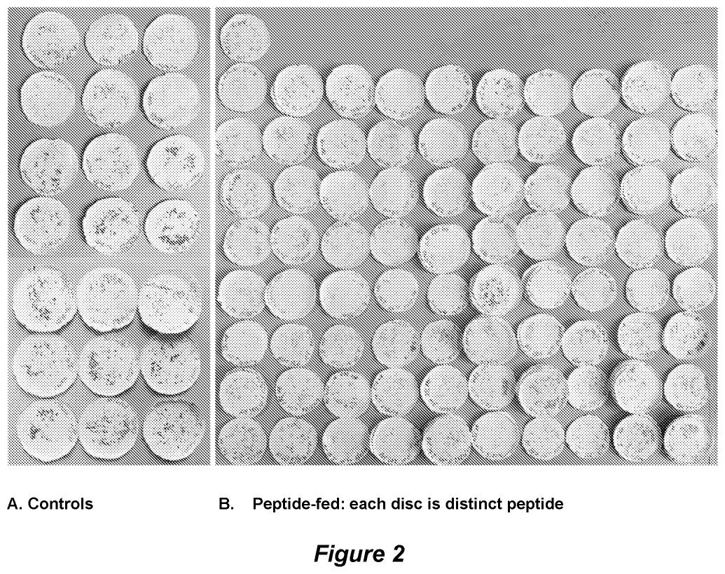

Capa peptide symptoms start with cessation of feeding in aphids: Overall, visual examination of honeydew production indicates that aphids fed significantly less on many capa peptides compared to controls at 24 hours, FIG. 2. Comparison of purple stain in controls to treated aphids, show there is very little colour on many membranes where peptides were added.

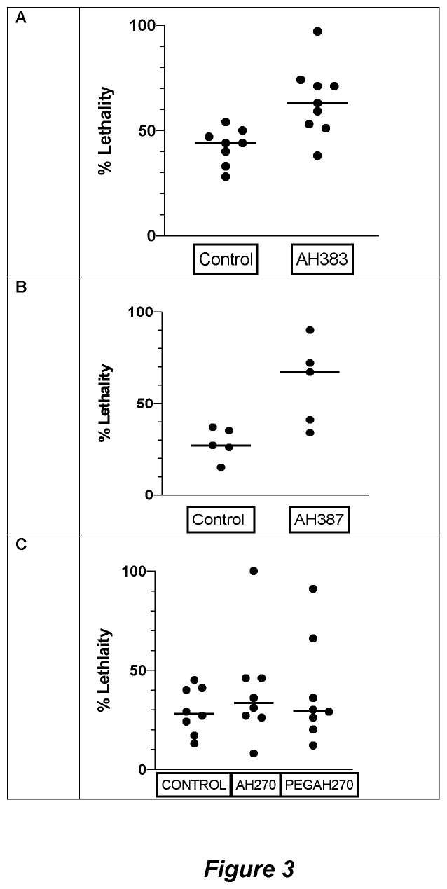

Capa peptides cause mortality in M. persicae via feeding: At 120 hours, AH383 and AH387 peptides induce ˜60% mean (and median) lethality. Although mean lethality is lower with AH270/PEGAH270 (40%), 100% lethality is observed in one sample of 30 insects (AH270); and 90% in at least one sample of each of AH383, AH387 and PEGAH270 (FIG. 3). Note that PEGAH270 is a pegylated form of peptide AH270.

### Example 3

### Topical Application by Spraying

Aphids were exposed to test peptides in the absence of additional external stress conditions.

Brassica rapa (Chinese cabbage; Wong Bok) were infested with 30 adult Myzus persicae aphids per plant. Aphids were left at least 2 hours to settle and begin feeding from the host plant.

Spraying took place inside a designated spray room. To ensure spray tracking, all sprayed solutions had amaranth dye added.

Potter Spray Tower (Burkard Manufacturing) was ‘primed’ by spraying 1000 μl of liquid coating the inside of the tower. Vehicle spray only (Croda ATPlus UEP 100 LQ-(CQ) 0.1% v/v) was used as a control.

Imidacloprid was also used as a positive control (28.3 μM), data not shown. Imidacloprid was always applied last and via a second, separate, Potter Tower, to prevent any possibility of stray pesticide being left inside the tower and contaminating a test peptide-applied plant.

Spray volumes for all solutions were 3000 μl. The 6.9 mm spray head was loaded with 3000 μl of a 1×10⁻⁵M peptide solution diluted in ATPlus 0.1%. After spraying was completed the plant was allowed to rest on the spray platform for 30 seconds to allow settling of the sprayed chemical.

During this spray process, due to the low pressure of the air stream, no aphids were observed to be dislodged from the plant.

Post peptide application, each condition was placed into its own individual Bugdorm (Watkins and Doncaster, 44545), to prevent repulsed or displaced aphids moving from one condition to another. Numbers of alive and dead aphids on the plant were counted 48 hours post spray, and the presence of any fresh nymphs noted. Plants were watered prior to spraying but not afterwards to eliminate the possibility of drowning any aphids present or washing off the sprayed liquid.

Post spraying, the spray head was filled with over 3000 μl of 70% ethanol and sprayed until empty. The spray head was carefully removed and rinsed with 70% ethanol as some amaranth dye was observed on the spray head. The inside of the tower was further cleaned by spraying 70% ethanol around the top and allowing it to drain down inside. The tower was then cleaned thoroughly by passing blue roll down from the top and up from the bottom of the tower. The spray platform is temporarily removed to allow access. The Potter Towers are cleaned between each use of peptide and at the end of experiments.

The vehicle control group was used to determine baseline lethality. Results for neuropeptide analogues were calculated as % lethality in the remaining population after adjustment for the baseline. These results are shown in Tables 7 and 8 below.

### Example 4

### Topical Application by Spraying

Aphid spray experiments were further conducted with selected peptides and compared with conventional insecticides (Imadocloprid; Spiroteramat) under different conditions: using an Airbrush or Potter Tower as explained in example 3; with aphid post-spray or pre-spray populated plants; and various Croda formulations (including Tweens) to determine conditions for the spray experiments.

Tests were conducted with 30 adult aphids per plant or per leaf, with 3 plants/leaves per treatment (90 aphids).

Post peptide application, each condition was placed into its own individual Bugdorm (Watkins and Doncaster, 44545), to prevent repulsed or displaced aphids moving from one condition to another. Numbers of alive and dead aphids on the plant were counted 120 hours post spray, and the presence of any fresh nymphs noted.

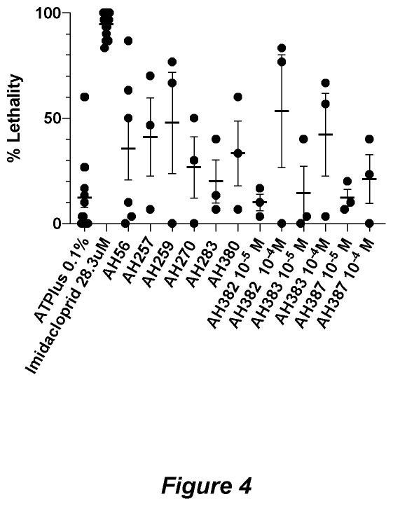

These Potter Tower assays indicate kill rate of ˜50%, and suggest that up to 80% kill is possible, FIG. 4.

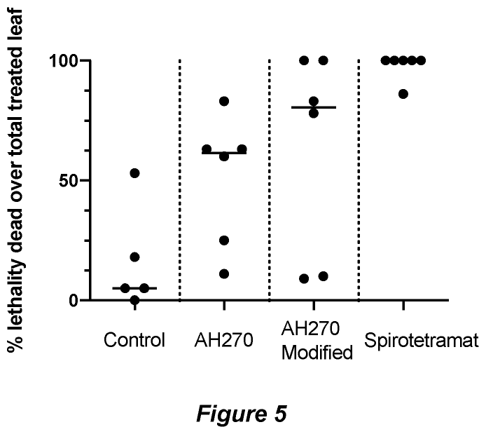

Further analysis of lethality considering only treated leaves was conducted. Data for 120 hours post-treatment at 10⁻³ M concentration of peptide indicate that aphids are killed effectively when they remain on treated leaves (FIG. 5).

### Example 5

### Peptide Stability Studies

Enzyme digests were performed using aminopeptidase, endopeptidase, carboxypeptidase, or Drosophila melanogaster Malpighian tubule tissue extract. Peptide digests were performed in triplicate alongside ‘mock’ digests, where protease was substituted with water to determine peptide stability in aqueous media at 37° C.

Several peptides were tested, including 1st generation AH56 and 3rs generation AH259. These are biostable in the presence of purified proteases for >24 hours; and for >3 hours to insect tissue extracts which are enriched for proteases.

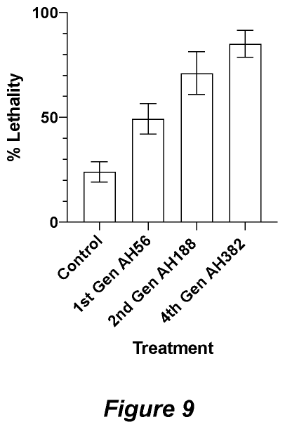

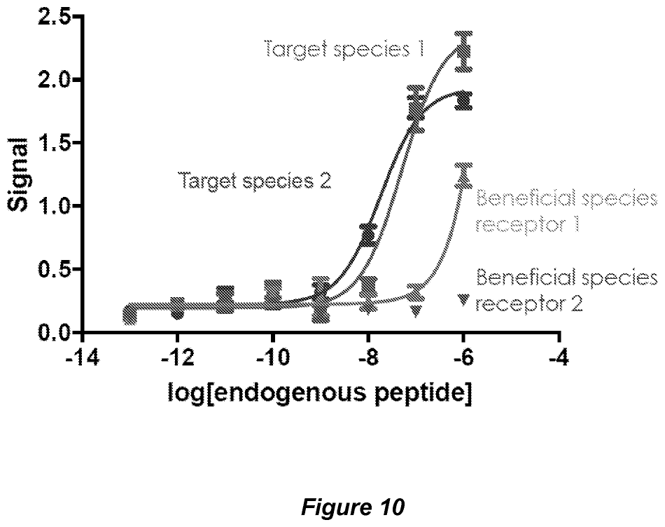

In vivo larval feeding assay tests (see FIG. 9), 72 hour treatment of insect lethality using ‘1st generation’ AH56, 2nd generation (AH188) and 4th generation (AH382) peptides shows increasing efficacy from 1st-4th generation, suggesting increased efficacy due to increased stability of later generation peptides (FIG. 9).

### Example 6

### Measurement of Intracellular Ca²⁺ in S2 Cells

Drosophila melanogaster S2 cells, cultured under standard conditions (1) were transiently transfected with the apoaequorin ORF (Radford J C, Davies S A, Dow J A. Systematic G-protein-coupled receptor analysis in Drosophila melanogaster identifies a leucokinin receptor with novel roles. J Biol Chem. 2002; 277:38810-38817) and a receptor ORF construct, and expression induced using CuSO4. Transfected S2 cells were harvested and incubated with 2.5 μM coelenterazine in the dark at RT for 1-2 h as described (ibid). 25,000 cells were then placed in 135 μl Schneider's medium supplemented with 10% FCS in a well of a white polystyrene 96-well plate (Berthold Technologies). Bioluminescence recordings were carried out using a Mithras LB940 automated 96-well plate reader (Berthold Technologies) and MikroWin software. 15 μl of each of different peptides were applied to final concentrations as required.

Peptides were tested at 10⁻⁷M.

At the end of each recording samples were disrupted by the addition of 100 μl lysis solution, and the [Ca²+] concentrations calculated as previously described (Rosay P, Davies S A, Yu Y, Sozen A, Kaiser K, et al. Cell-type specific calcium signalling in a Drosophila epithelium. J Cell Sci. 1997; 110:1683-1692).

For the Capa1 and Capa2 peptides, the relevant native control peptide (D. melanogaster Capa1 or Capa2 respectively) was used as a control. Control agonist activity was normalised to 100% and activity of neuropeptide analogues is expressed relative to that. Results are shown in Tables 7 and 8 below.

### Example 7

Measurement of intracellular Ca²⁺ in mammalian cells

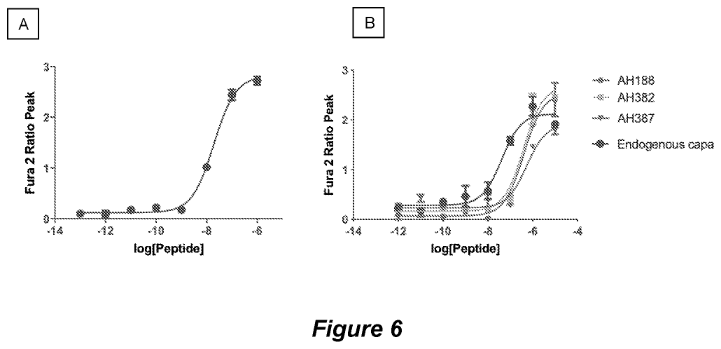

To further develop the molecular screening platform for mode of action studies described in example 5, where S2 cells were transiently transfected with Drosophila melanogaster capa GPCR, capaR, (activated by ‘FPRV’ peptides) and apoaequorin and then assayed for peptide-stimulated calcium (Ca2+) signal (Terhzaz et al., 2012) and compared to native capa-stimulated Ca2+. Stable mammalian cell lines were generated for D. suzukii with CapaRs datamined receptor sequences, cloned and expressed. Results from testing of native capa and candidate peptides against species-specific receptors are shown in FIG. 6 a showing activation of D. suzukii CapaR by endogenous capa peptide; and differential activation by different peptide candidates (FIG. 6 b).

### Example 8

Drosophila suzukii larval feeding

Peptides were tested at concentrations ranging from [10⁻⁴ M] to [10⁻⁷ M] in a final volume of 200 μl of 0.8% agarose containing 0.09% m-Cresol purple pH marker dye (Sigma) and 5% sucrose (Sigma).

Agarose was melted in dH₂O, before addition of sucrose and dye. The agarose/dye solution was then allowed to cool to 60° C. 96 well plates (flat-bottomed 3596 TC-plates; Corning) containing 20 μl volumes of neuropeptide per well were prepared, and 180 μl agarose/dye solution dispensed into each well via a Distriman repetitive micro-pipettor (Gilson). Controls contained 20 μl dH₂O as a ‘sham’ treatment with 180 μl of the 0.8% agarose/dye solution. Plates were agitated at 800 rpm, to ensure mixing, at 60° C. (Eppendorf Thermomixer C). Plates were then allowed to cool, and agarose/dye solution solidify, prior to use.

Each peptide was assayed with a minimum of 7 to maximum 9 late L2/early L3 Spotted wing Drosophila, Drosophila suzukii, (feeding) larvae per well, minimum n=8 wells per peptide concentration and a minimum n=12 control wells per plate. The selected larvae were first washed 2× in ice cold Drosophila Schneider's liquid medium (ThermoFisher) before insertion into wells to remove extraneous food. After complete insertion of all larvae the plates were covered with breathable sealing membrane for multi-well plates (Sigma).

Larvae were assayed for lethality after 48 hr exposure at 21° C. and findings recorded. During examination the assay plate was kept on ice, to reduce larval locomotor activity (wandering), and larvae in each well were examined using a binocular stereomicroscope (Zeiss).

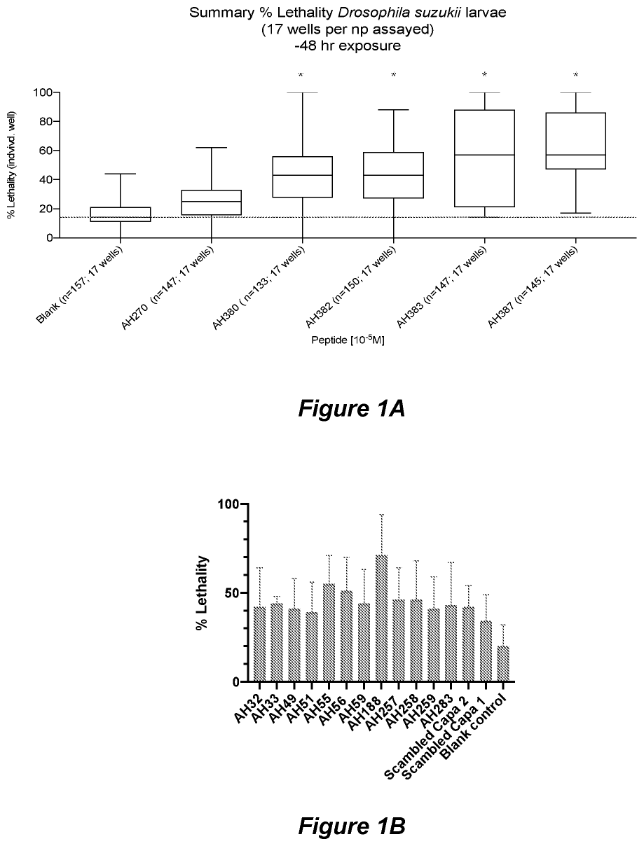

The water control group was used to determine baseline lethality. Results for neuropeptide analogues were calculated as % lethality in the remaining population after adjustment for the baseline. Results are shown in Tables 7 and 8 below, and in FIG. 1.

### Example 9

Drosophila suzukii Larval Feeding

Further larval feeding assays with D. suzukii larvae were conducted as explained above in example 7. A total of 88 peptides were tested against D. suzukii larvae in 96 well plates at 10⁻⁵ M in a final volume of 200 μl of 0.8% agarose containing 0.09% m-Cresol purple pH marker dye (Sigma) and 5% sucrose (Sigma). Controls contained dH₂O (18% lethality) or native Capa-2 peptide (38% lethality) as a ‘sham’ treatment. Each peptide was assayed with a minimum of 7 to maximum 9 late L2/early L3 larvae per well, minimum n=8 wells per peptide concentration (minimum 56 larvae) and a minimum n=12 control wells (minimum 84 larvae) per plate. Larvae were assayed for lethality after 48 hr and/or 72 h exposure at 21° C. using a binocular stereomicroscope and findings recorded. Candidate peptides were also tested between 10-4 and 10-7 M (dose response curve).

Candidate peptides for D. suzukii with 70%-90% lethality are indicated below, in Table 6. Several candidates also showed efficacy at 60-70% and between 50-60% lethality.

TABLE 6

| Peptide | % Lethality ± SEM |
|---|---|
| AH382 | 90 + 4 |
| AH383 | 80 ± 3 |
| AH270/PEGAH270 | 75 ± 5/77 ± 4 |
| AH188 | 71 ± 6 |

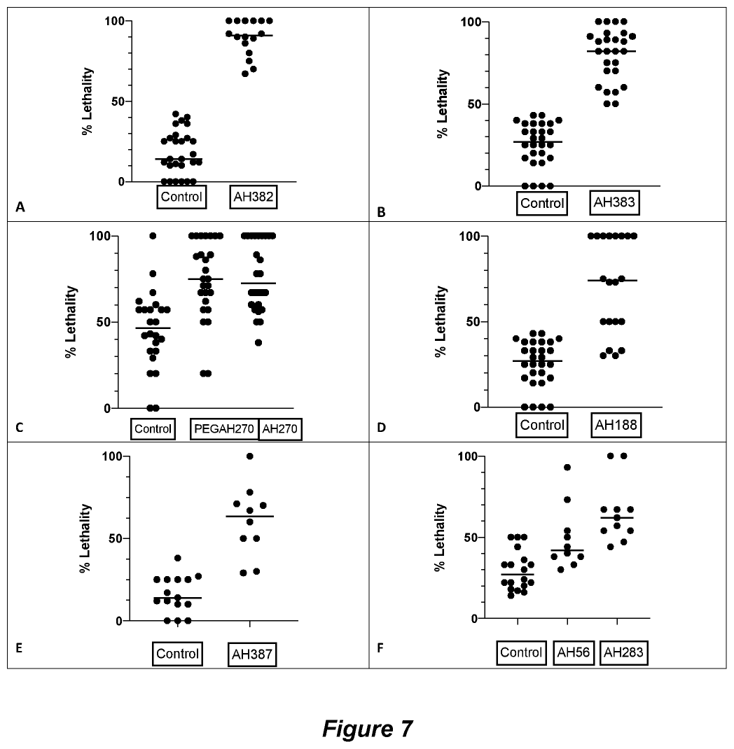

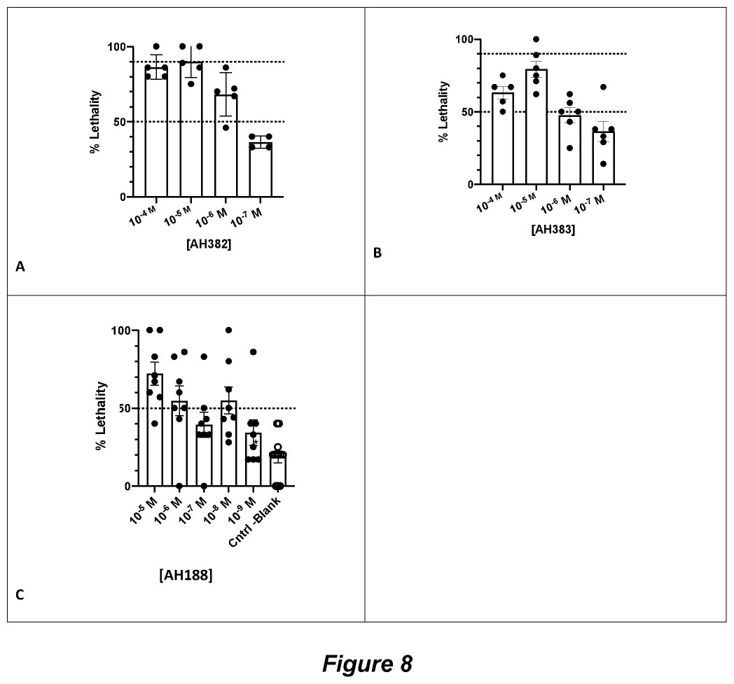

FIG. 7 shows the in vivo lethality efficacy data after 72 hour treatment of larvae with the indicated Capa peptides, except for AH270/PEGAH270 treatments which were 120 hour treatments. FIG. 8 shows the calculated Dose-response curves for some peptides: AH382, AH383, and AH188.

### Example 10

### Bumblebee Acute Oral Toxicity Test

Oral toxicity against bumblebees (Bombus terrestris) was determined as previously described:

OECD (2017), Test No. 247: Bumblebee, Acute Oral Toxicity Test, OECD Guidelines for the Testing of Chemicals, Section 2, OECD Publishing, Paris, https://doi.org/10.1787/9789264284128-en.

Example data are provided for peptides, AH56, AH257, AH259 in Tables 4, 5 below. Treatment conditions were as follows:
(i) 50% sucrose
(ii) 50% sucrose, 0.1% Croda ATPlus
(iii) 50% sucrose, 0.1% ATPlus, 1×10⁻⁵ M AH peptide
(iv) 50% sucrose, 0.1% ATPlus, 28.3 uM Imidacloprid
(v) 50% sucrose, No bee control, evaporation

Data were acquired for: feeding behaviour—weight of liquid consumed pre- and post-treatment; lethality (days).

Control show 100% survival after 4 days; whereas imidacloprid-fed bees showed 100% lethality on day 1. No lethal effect was observed for AH peptides AH56, AH257 or AH259. There was no negative impact on feeding behaviour from capa peptides. Acute bumblebee toxicity was also assessed for other peptide candidates including: AH270, PEG270. Overall, capa peptides are bee safe. Capa peptide safety for other beneficial species has also been demonstrated (Gui et al., Pest Management Science, 2020).

### Results

TABLE 7
CAPA2 analogues:

| | CapaR | | | | | | | | | | |
|---|---|---|---|---|---|---|---|---|---|---|---|
| | Ca²⁺ | | | | | | | | | | |
| | Agonist | Feeding | | | | | | | | Spray | |
| | response¹ | D. suzukii² | | | Feeding Aphid² | | | | | Aphid² | |
| | Concentration (M) | | | | | | | | | | |
| Compound | 10⁻⁷ | 10⁻³ | 10⁻⁴ | 10⁻⁵ | 10⁻³ | 10⁻⁴ | 10⁻⁵ | 10⁻⁶ | 10⁻⁷ | 10⁻⁴ | 10⁻⁵ |
| AH56 | 135 | | | 51 ± | | | 30 | | | 41 ± | 35 ± |
| | | | | 24 | | | | | | 11 | 15 |
| AH257 | 85 | | | 46 ± | | | 25 | | | 21 ± | 41 ± |
| | | | | 18 | | | | | | 7 | 18 |
| AH259 | 12 | | | 41 ± | | 18 | 13 ± | 24 | 19 | 47 ± | 42 ± |
| | | | | 18 | | | 1 | | | 30 | 30 |
| AH283 | 21 | | | 43 ± | | 34 ± | 26 ± | 27 ± | 20 ± | 30 ± | 20 ± |
| | | | | 24 | | 7 | 12 (51 ± | 11 | 3 | 25 | 10 |
| | | | | | | | 7@ 72 h) | | | | |
| AH270 | 85 | | | 27 ± | | | 43 ± | | | | 27 ± |
| | | | | 18 | | | 11 (60 h) | | | | 15 |

¹activity normalised to that of native Capa2 (designated 100%).
²% lethality in population after adjustment for baseline.

TABLE 8
CAPA1 analogues:

| | CapaR | | | | | | | | | | |
|---|---|---|---|---|---|---|---|---|---|---|---|
| | Ca²⁺ | | | | | | | | | | |
| | Agonist | Feeding | | | | | | | | Spray | |
| | response¹ | D. suzukii² | | | Feeding Aphid² | | | | | Aphid² | |
| Compound | Concentration (M) | | | | | | | | | | |
| ID | 10⁻⁷ | 10⁻³ | 10⁻⁴ | 10⁻⁵ | 10⁻³ | 10⁻⁴ | 10⁻⁵ | 10⁻⁶ | 10⁻⁷ | 10⁻⁴ | 10⁻⁵ |
| AH188 | 64 | | | 71 ± | | 23 | 23 ± | 22 | 17 | 2 ± | |
| | | | | 23 | | | 1 (33%@72 h) | | | 4 | |
| AH380 | 14 | | | 45 ± | | | 12 ± | | | | 33 ± |
| | | | | 27 | | | 8 | | | | 15 |
| AH382 | 36 | | | 43 ± | | | 12 ± | | | 49 ± | 10 ± |
| | | | | 26 | | | 8 | | | 31 | 4 |
| AH383 | 26 | | | 56 ± | | | 10 ± | | | 42 ± | 13 ± |
| | | | | 34 | | | 11 | | | 20 | 13 |
| AH387 | 17 | | | 62 ± | | | 14 ± | | | | 12 |
| | | | | 24 | | | 6 (35 ± | | | | |
| | | | | | | | 8@60 h) | | | | |

¹activity normalised to that of native Capal (designated 100%).
²% lethality in population after adjustment for baseline.

Compounds AH56, AH257, AH259, and AH270/PEGAH270 were determined to be safe towards B. terrestris in the bumblebee oral toxicity assay.

Table 9 below: Data for individual bees tested with AH257 and imidacloprid and controls. ‘dead?’ column where A=alive at 4 days; and numbers e.g. 1 or 2 indicates death on those days. Feeding data (summarised in Feeding impacted), where positive indicates no detrimental effect; negative e.g., for imidacloprid indicates reduced feeding.

| | | | | | | grams/day liquid |
|---|---|---|---|---|---|---|
| | Pre peptide | | | | | +ve feeding |
| % of test liquid consumed by group | | | | | | increased post pep |
| 1. 50% sucrose | | | weight sucrose | Est weight | | −ve feeding |
| | mass test | | consumed pre | sucrose consumed | | decreased post pep |
| Bee no. | consumed (g) | dead? | pre testing | post testing/day | | feeding impacted? |
| [symbol] | 0.0367 | A | 0. [symbol] | 0. [symbol] | | −0.0 [symbol] |
| [symbol] 0 | 0.0638 | A | 0. [symbol] | 0.3 [symbol] | | 0.0 [symbol] |
| 34 | 0.0032 | A | 0. [symbol] | 0. [symbol] | | −0.0 [symbol] |
| 29 | 0.0302 | A | 0.2970 | 0. [symbol] | | 0. [symbol] |
| 23 | 0.0453 | A | 0.29 [symbol] | 0.2 [symbol] 12 | | −0.0 [symbol] |
| 27 | 0.0448 | A | 0.2771 | 0. [symbol] | | 0.0 [symbol] |
| 32 | 0.0027 | A | 0. [symbol] | 0. [symbol] | | −0.0 [symbol] |
| 37 | 0.0490 | A | 0.2 [symbol] | 0. [symbol] | | 0.0 [symbol] |
| 41 | 0.0439 | A | 0. [symbol] | 0. [symbol] | | 0.0 [symbol] |
| 45 | 0.0377 | A | 0. [symbol] | 0. [symbol] | | −0.0 [symbol] |
| mean | | | [symbol] | [symbol] | total | [symbol] |
| 2. 50% sucrose, 0.1% ATPlus | | | | | | |
| [symbol] | 0.0 [symbol] | A | 0.2 [symbol] | 0.0 [symbol] | | −0. [symbol] |
| [symbol] | 0.0 [symbol] | A | 0.4 [symbol] | 0. [symbol] | | −0. [symbol] |
| [symbol] | 0.0 [symbol] | A | 0. [symbol] | 0. [symbol] | | 0. [symbol] |
| 20 | 0.0 [symbol] | A | 0.2810 | 0. [symbol] | | 0.0 [symbol] |
| [symbol] | 0.0 [symbol] | A | 0. [symbol] | 0. [symbol] | | 0.0 [symbol] |
| [symbol] | 0.0 [symbol] | A | 0. [symbol] | 0. [symbol] | | 0.0 [symbol] |
| [symbol] | 0.0 [symbol] | A | 0. [symbol] | 0. [symbol] | | 0. [symbol] |
| 3 [symbol] | 0.0 [symbol] | A | 0. [symbol] | 0. [symbol] | | 0. [symbol] |
| 42 | 0.0 [symbol] | A | 0. [symbol] | 0. [symbol] | | [symbol] 0.0 [symbol] |
| 48 | 0.0 [symbol] | [symbol] | 0. [symbol] | 0. [symbol] | | −0.0 [symbol] |
| mean | | | [symbol] | [symbol] | total | [symbol] |
| 3. 50% sucrose, 0.1% ATPlus, 257 × 10 − 5M AH | | | | | | |
| 2 | 0.0 [symbol] | A | 0. [symbol] | 0. [symbol] | | 0.0 [symbol] |
| 5 | 0.0 [symbol] | [symbol] | 0. [symbol] | 0. [symbol] | | −0.0 [symbol] |
| 12 | 0.0 [symbol] | A | 0. [symbol] | 0. [symbol] | | 0.0 [symbol] |
| [symbol] | 0.0 [symbol] | A | 0. [symbol] | 0. [symbol] | | 0.0 [symbol] |
| 21 | 0.0 [symbol] | A | 0. [symbol] | 0. [symbol] | | −0.0 [symbol] |
| [symbol] | 0.0 [symbol] | A | 0. [symbol] | 0. [symbol] | | −0.0 [symbol] |
| 30 | 0.0 [symbol] | A | 0. [symbol] | 0. [symbol] | | −0.0 [symbol] |
| 35 | 0.0 [symbol] | A | 0. [symbol] | 0. [symbol] | | 0. [symbol] |
| [symbol] | 0.0 [symbol] | A | 0. [symbol] | 0. [symbol] | | 0. [symbol] |
| [symbol] | 0.0 [symbol] | A | 0. [symbol] | 0. [symbol] | | 0. [symbol] |
| [symbol] | 0.0 [symbol] | A | 0. [symbol] | 0. [symbol] | | 0. [symbol] |
| mean | | | [symbol] | [symbol] | total | [symbol] |
| 4. 50% sucrose, 0.1% ATPlus, 28.3 uM [symbol] | | | | | | |
| 3 | 0.0 [symbol] | A | 0. [symbol] | 0.0822 | | −0. [symbol] |
| [symbol] | 0.0 [symbol] | [symbol] | 0. [symbol] | 0.0 [symbol] | | −0. [symbol] |
| [symbol] | 0.0 [symbol] | [symbol] | 0. [symbol] | 0.0 [symbol] | | −0. [symbol] |
| 18 | 0.0 [symbol] | [symbol] | 0. [symbol] | 0.0000 | | −0. [symbol] |
| 22 | 0.0 [symbol] | [symbol] | 0. [symbol] | 0.0000 | | −0. [symbol] |
| 25 | 0.0 [symbol] | [symbol] | 0. [symbol] | 0.0 [symbol] | | −0. [symbol] |
| [symbol] | 0.0 [symbol] | [symbol] | 0. [symbol] | 0. [symbol] | | 0.0 [symbol] |
| [symbol] | 0.0 [symbol] | [symbol] | 0. [symbol] | 0.0 [symbol] | | −0. [symbol] |
| [symbol] | 0.0 [symbol] | [symbol] | 0. [symbol] | 0. [symbol] | | 0. [symbol] |
| [symbol] | 0.0 [symbol] | [symbol] | 0. [symbol] | 0.0 [symbol] | | −0. [symbol] |
| [symbol] | 0.0 [symbol] | [symbol] | 0. [symbol] | 0.0000 | | −0. [symbol] |
| mean | | | [symbol] | [symbol] | total | [symbol] |

T-test (paired, two tailed)
sucrose consumed Pre vs Post
1)sucrose control 0. [symbol] No sig effect
2)ATPlus vehicle 0.28 No sig effect
3)257 × 10 − 5M 0.17 No sig effect
4) [symbol] control 0.0 [symbol] Feeding sig reduced [symbol]
indicates data missing or illegible when filed

Table 10 below: Data for individual bees tested with AH259 and imidacloprid and controls. ‘dead?’ column where A=alive at 4 days; and numbers e.g. 1 or 2 indicates death on those days. Feeding data (summarised in Feeding impacted), where positive indicates no detrimental effect; negative e.g., for imidacloprid indicates reduced feeding.

| | | | | | | grams/day liquid |
|---|---|---|---|---|---|---|
| | Pre peptide | | | | | +ve feeding |
| % of test liquid consumed by group | | | | | | increased post pep |
| 1. 50% sucrose | | | weight | Est weight | | −ve feeding |
| | mass test | | sucrose consumed | sucrose consumed | | decreased post pep |
| Bee no. | consumed (g) | dead? | pre testing | post testing/day | | Feeding impacted? |
| 4 | 0.0367 | A | 0.2063 | 0.1570 | | −0.0493 |
| 10 | 0.0434 | A | 0.3311 | 0.3525 | | 0.0214 |
| 14 | 0.0032 | A | 0.1563 | 0.1039 | | −0.0524 |
| 19 | 0.0302 | A | 0.2970 | 0.4060 | | 0.1090 |
| 23 | 0.0453 | A | 0.2984 | 0.2212 | | −0.0772 |
| 27 | 0.0448 | A | 0.2771 | 0.3594 | | 0.0823 |
| 32 | 0.0027 | A | 0.2828 | 0.2227 | | −0.0601 |
| 37 | 0.0460 | A | 0.2243 | 0.2533 | | 0.0290 |
| 41 | 0.0430 | A | 0.2362 | 0.3049 | | 0.0687 |
| 45 | 0.0377 | A | 0.3689 | 0.3408 | | −0.0281 |
| mean | | | [symbol] | [symbol] | total | [symbol] |
| 2. 50% sucrose, 0.1% ATPlus | | | | | | |
| 5 | 0.0104 | A | 0.2416 | 0.0417 | | −0.1999 |
| 11 | 0.0453 | A | 0.4143 | 0.4050 | | −0.0093 |
| 15 | 0.0454 | A | 0.2127 | 0.3896 | | 0.1769 |
| 20 | 0.0269 | A | 0.2810 | 0.3225 | | 0.0415 |
| 24 | 0.0057 | A | 0.1648 | 0.2543 | | 0.0895 |
| 28 | 0.0479 | A | 0.1461 | 0.2234 | | 0.0773 |
| 34 | 0.0481 | A | 0.1784 | 0.3604 | | 0.1820 |
| 38 | 0.0443 | A | 0.2818 | 0.4020 | | 0.1202 |
| 42 | 0.0070 | A | 0.1387 | 0.1341 | | −0.0046 |
| 4 [symbol] | 0.0045 | 2 | 0.1554 | 0.1016 | | −0.0538 |
| mean | | | [symbol] | [symbol] | total | [symbol] |
| 3. 50% sucrose, 0.1% ATPlus, 257 × 10 − 5M AH | | | | | | |
| 2 | 0.0425 | A | 0.3887 | 0.4789 | | 0.0902 |
| 6 | 0.0118 | 2 | 0.1705 | 0.0824 | | −0.0881 |
| 12 | 0.0429 | A | 0.1841 | 0.2612 | | 0.0770 |
| 17 | 0.0442 | A | 0.3444 | 0.3450 | | 0.0006 |
| 21 | 0.0023 | A | 0.1694 | 0.1265 | | −0.0429 |
| 25 | 0.0078 | A | 0.4169 | 0.4161 | | −0.0008 |
| 30 | 0.0272 | A | 0.3105 | 0.2912 | | −0.0193 |
| 35 | 0.0425 | A | 0.1638 | 0.3677 | | 0.2039 |
| 39 | 0.0203 | A | 0.1951 | 0.2298 | | 0.0347 |
| 43 | 0.0379 | A | 0.2021 | 0.3016 | | 0.0995 |
| 47 | 0.0320 | A | 0.2452 | 0.2869 | | 0.0416 |
| mean | | | [symbol] | [symbol] | total | [symbol] |
| 4. 50% sucrose, 0.1% ATPlus, 28.3 uM [symbol] | | | | | | |
| 3 | 0.0114 | A | 0.2001 | 0.0822 | | −0.1179 |
| 8 | 0.0279 | 1 | 0.1704 | 0.0495 | | −0.1209 |
| 13 | 0.0285 | 1 | 0.1506 | 0.0000 | | −0.1306 |
| 18 | 0.0408 | 1 | 0.3025 | 0.0000 | | −0.3025 |
| 22 | 0.0251 | 1 | 0.1495 | 0.0000 | | −0.1495 |
| 26 | 0.0413 | 1 | 0.1974 | 0.0562 | | −0.1412 |
| 31 | 0.0023 | A | 0.1501 | 0.2205 | | 0.0704 |
| 36 | 0.0391 | 1 | 0.3396 | 0.0000 | | −0.3396 |
| 40 | 0.0038 | 1 | 0.2439 | 0.4394 | | 0.1955 |
| 44 | 0.0082 | 1 | 0.2914 | 0.0000 | | −0.2914 |
| 48 | 0.0425 | 1 | 0.3507 | 0.0000 | | −0.3507 |
| mean | | | [symbol] | [symbol] | total | [symbol] |

T-test (paired, two tailed)
sucrose consumed Pre vs Post
1)sucrose control 0.84 No sig effect
2)ATPlus vehicle 0.28 No sig effect
3)257 × 10 − 5M 0.17 No sig effect
4) [symbol] control 0.01 Feeding sig reduced [symbol]
indicates data missing or illegible when filed

Table 11 below: Data for individual bees tested with AH270 and AHPEG270 and imidacloprid and controls. ‘dead?’ column where A=alive at 4 days; and numbers e.g. 1 or 2 indicates death on those days. Feeding data (summarised in Feeding impacted), where positive indicates no detrimental effect; negative e.g., for imidacloprid indicates reduced feeding.

| | | Starting bees | Bees died | % lethality | Weights in g | | |
|---|---|---|---|---|---|---|---|
| Testing regime | 1) Sucrose 50% | | | | | | |
| | | liquid | | | | | |
| | | consumed | | | | | |
| | | (weight) | | | liquid | | |
| | | Liquid pre | Liquid | | consumed | Test | |
| | | exposure | post | | per day post | liquid | |
| Bee id | Weight of Bee | (24 hrs) | exposure | days alive | exposure | consumed | n= |
| 5 | 0.1994 | 0.1861 | 0.3969 | 4 | 0.0992 | 0.0411 | 10 |
| 11 | 0.1886 | 0.1159 | 1.0582 | 4 | 0.2646 | 0.0256 | |
| 26 | 0.2372 | 0.1358 | 1.4669 | 4 | 0.3667 | 0.0443 | |
| 32 | 0.1923 | 0.2259 | 1.3489 | 4 | 0.3372 | 0.0465 | |
| 37 | 0.2131 | 0.1335 | 1.3824 | 4 | 0.3456 | 0.0445 | |
| 43 | 0.1985 | 0.2624 | 1.9386 | 4 | 0.4847 | 0.0033 | |
| 49 | 0.2139 | 0.1719 | 0.3532 | 4 | 0.0883 | 0.0038 | |
| 55 | 0.1575 | 0.3753 | 1.8029 | 4 | 0.4507 | 0.0462 | |
| 63 | 0.1963 | 0.1562 | 0.3679 | 4 | 0.0920 | 0.0034 | |
| 69 | 0.1459 | 0.1366 | 0.9956 | 4 | 0.2489 | 0.0461 | |
| Mean | 0.1943 | 0.1900 | 1.1110 | | 0.2778 | 0.0305 | |
| SEM | 0.0084 | 0.0251 | 0.1848 | | 0.0462 | 0.0062 | |
| | | Starting bees | Bees died | % lethality | | | |
| Testing regime | 2) ATPlus 0.1%, Sucrose 50% | | | | | | |
| | | liquid | | | liquid | | |
| | | consumed | Liquid | | consumed | Test | |
| | | Liquid pre | post | | per day post | liquid | |
| Bee id | Weight of Bee | exposure | exposure | days alive | exposure | consumed | n= |
| 6 | 0.2342 | 0.3414 | 1.0260 | 4 | 0.2565 | 0.0008 | 10 |
| 16 | 0.2481 | 0.1779 | 0.8851 | 4 | 0.2213 | 0.0443 | |
| 27 | 0.1791 | 0.1268 | 0.3348 | 4 | 0.0837 | 0.0134 | |
| 33 | 0.2114 | 0.1980 | 1.6954 | 4 | 0.4239 | 0.0469 | |
| 39 | 0.1828 | 0.3097 | 0.9270 | 4 | 0.2318 | 0.0465 | |
| 44 | 0.1676 | 0.3151 | 1.5053 | 4 | 0.3763 | 0.0417 | |
| 51 | 0.1823 | 0.2922 | 1.2769 | 4 | 0.3192 | 0.0461 | |
| 56 | 0.2231 | 0.1741 | 0.3826 | 4 | 0.0957 | 0.0412 | |
| 64 | 0.1408 | 0.2220 | 1.0775 | 4 | 0.2694 | 0.0069 | |
| 70 | 0.1621 | 0.1911 | 0.3715 | 4 | 0.0929 | 0.0040 | |
| Mean | 0.1932 | 0.2348 | 0.9482 | | 0.2371 | 0.2918 | |
| SEM | 0.0109 | 0.0233 | 0.1503 | | 0.0376 | 0.0063 | |
| | | Starting bees | Bees died | % lethality | | | |
| Testing regime | 3) AH270 1 × 10 − 5M, Sucrose 50%, ATPlus 0.1% | | | | | | |
| | | liquid | | | liquid | | |
| | | consumed | Liquid | | consumed | Test | |
| | | Liquid pre | post | | per day post | liquid | |
| Bee id | Weight of Bee | exposure | exposure | days alive | exposure | consumed | n= |
| 1 | 0.2625 | 0.2347 | 0.9262 | 4 | 0.2316 | 0.0064 | 10 |
| 7 | 0.1942 | 0.1177 | 0.7170 | 4 | 0.1793 | 0.0077 | |
| 18 | 0.2614 | 0.2442 | 1.8815 | 4 | 0.4704 | 0.0461 | |
| 28 | 0.1475 | 0.1118 | 0.8535 | 4 | 0.2134 | 0.0065 | |
| 34 | 0.1764 | 0.1704 | 0.6307 | 4 | 0.1577 | 0.0060 | |
| 40 | 0.1407 | 0.1540 | 0.4568 | 4 | 0.1142 | 0.0060 | |
| 45 | 0.1642 | 0.1245 | 0.3007 | 4 | 0.0752 | 0.0470 | |
| 52 | 0.2165 | 0.1372 | 0.5227 | 4 | 0.1307 | 0.0246 | |
| 57 | 0.1808 | 0.2345 | 1.1365 | 4 | 0.2841 | 0.0033 | |
| 65 | 0.1934 | 0.1788 | 0.3679 | 4 | 0.0920 | 0.0064 | |
| Mean | 0.1938 | 0.1708 | 0.7794 | | 0.1949 | 0.0160 | |
| SEM | 0.0134 | 0.0161 | 0.1477 | | 0.0369 | 0.0054 | |
| | | Starting bees | Bees died | % lethality | | | |
| Testing regime | 4) AHPEG270 1 × 10 − 5M, Sucrose 50%, ATPlus 0.1% | | | | | | |
| | | liquid | | | liquid | | |
| | | consumed | Liquid | | consumed | Test | |
| | | Liquid pre | post | | per day post | liquid | |
| Bee id | Weight of Bee | exposure | exposure | days alive | exposure | consumed | n= |
| 3 | 0.1749 | 0.2904 | 1.5744 | 4 | 0.3936 | 0.0405 | 10 |
| 9 | 0.2355 | 0.3361 | 0.7150 | 4 | 0.1788 | 0.0467 | |
| 20 | 0.1942 | 0.2563 | 0.4326 | 4 | 0.1082 | 0.0460 | |
| 29 | 0.1559 | 0.1049 | 0.2731 | 4 | 0.0683 | 0.0092 | |
| 35 | 0.2269 | 0.4520 | 1.1763 | 4 | 0.2941 | 0.0456 | |
| 41 | 0.1760 | 0.3211 | 1.8061 | 4 | 0.4515 | 0.0440 | |
| 46 | 0.1328 | 0.2115 | 0.5676 | 4 | 0.1419 | 0.0437 | |
| 53 | 0.1664 | 0.2589 | 1.5696 | 4 | 0.3924 | 0.0451 | |
| 58 | 0.1745 | 0.2717 | 0.6889 | 4 | 0.1722 | 0.0476 | |
| 67 | 0.1584 | 0.2794 | 0.0102 | 0 (approx 1 hr) | 0.0102 | 0.0053 | |
| Mean | 0.1796 | 0.2782 | 0.8814 | | 0.2211 | 0.0374 | |
| SEM | 0.0100 | 0.0281 | 0.1941 | | 0.0481 | 0.0051 | |
| | | Starting bees | Bees died | % lethality | | | |
| Testing regime | 5) Spirotetramat 25 uM | | | | | | |
| | | liquid | | | liquid | | |
| | | consumed | Liquid | | consumed | Test | |
| | | Liquid pre | post | | per day post | liquid | |
| Bee id | Weight of Bee | exposure | exposure | days alive | exposure | consumed | n= |
| 4 | 0.1998 | 0.2036 | 0.7878 | 4 | 0.1970 | 0.0037 | 10 |
| 10 | 0.2090 | 0.2300 | 1.2332 | 4 | 0.3083 | 0.0017 | |
| 25 | 0.1687 | 0.1432 | 0.2963 | 4 | 0.0741 | 0.0042 | |
| 31 | 0.2323 | 0.2876 | 1.7853 | 4 | 0.4463 | 0.0461 | |
| 36 | 0.2014 | 0.2462 | 1.2062 | 4 | 0.3016 | 0.0057 | |
| 42 | 0.1935 | 0.3499 | 2.1130 | 4 | 0.5283 | 0.0046 | |
| 47 | 0.2180 | 0.3417 | 0.6196 | 4 | 0.1549 | 0.0473 | |
| 54 | 0.2167 | 0.2351 | 0.7732 | 4 | 0.1933 | 0.0029 | |
| 62 | 0.2045 | 0.2159 | 1.3842 | 4 | 0.3461 | 0.0031 | |
| 68 | 0.1927 | 0.2357 | 0.8924 | 4 | 0.2231 | 0.0223 | |
| Mean | 0.2037 | 0.2489 | 1.109 | | 0.2773 | 0.0142 | |
| SEM | 0.0055 | 0.0198 | 0.1741 | | 0.0435 | 0.0057 | |

Table 12 below: Data for individual bees tested with AH56 and controls.

| | Mass dish | Mass dish | Mass of | Volume | | Volume | |
|---|---|---|---|---|---|---|---|
| | before (g) | and Bee (g) | Bee (g) | remaining (ul) | BGD'd? | removed | |
| 20% sucrose only | | | | | | | |
| 1 | 34.534 | 34.758 | 0.224 | 175 | Y | 325 | |
| 2 | 34.655 | 34.884 | 0.229 | 270 | Y | 230 | |
| 3 | 34.602 | 34.920 | 0.318 | 110 | Y | 390 | |
| 4 | 34.355 | 34.617 | 0.262 | 186 | Y | 314 | |
| 5 | 34.623 | 35.020 | 0.397 | 160 | Y | 340 | |
| | | | 0.286 | 180.2 | | 319.8 | |
| 20% sucrose 0.1% ATPlus | | | | | | | |
| 1 | 34.688 | 34.935 | 0.247 | 167 | Y | 333 | |
| 2 | 34.848 | 35.08 | 0.232 | 148 | Y | 352 | |
| 3 | 34.674 | 34.995 | 0.321 | 410 | N | 90 | Rejected due |
| | | | | | | | to not feeding |
| 4 | 34.617 | 34.926 | 0.309 | 75 | Y | 425 | |
| 5 | 34.764 | 35.01 | 0.246 | 241 | Y | 259 | |
| | | | 0.259 | 157.75 | | 342.25 | |
| 20% sucrose 0.1% ATPlus AH56 | | | | | | | |
| 1 | 35.023 | 35.205 | 0.182 | 211 | Y | 289 | |
| 2 | 34.563 | 34.773 | 0.210 | 295 | Y | 205 | |
| 3 | 34.677 | 35.200 | 0.523 | 248 | Y | 252 | |
| 4 | 34.633 | 34.901 | 0.268 | 216 | Y | 284 | |
| 5 | 34.611 | 34.873 | 0.262 | 178 | Y | 322 | |
| | | | 0.289 | 229.6 | | 270.4 | |
| | | 20% sucrose | +ATPlus | +AH56 | | | T Test |
| | Vol remaining | 175 | 167 | 211 | | Sucrose vs ATPlus | 0.619203 |
| | | 270 | 148 | 295 | | Sucrose vs AH56 | 0.170915 |
| | | 110 | 75 | 248 | | ATPlus vs AH56 | 0.128712 |
| | | 186 | 241 | 216 | | | |
| | | 160 | | 178 | | | |

### REFERENCES

A number of publications are cited above in order to more fully describe and disclose the invention and the state of the art to which the invention pertains. Full citations for these references are provided below. The entirety of each of these references is incorporated herein.
1. Pimentel D, Acquay H, Biltonen M, Rice P, Silva M, Nelson J, Lipner V, Giordano S, Horowitz A and Damore M, Environmental and economic costs of pesticide use. Bioscience 42: 750-760 (1992).
2. Wilson C and Tisdell C, Why farmers continue to use pesticides despite environmental, health and sustainability costs. Ecol Econ 39: 449-462 (2001).
3. Altstein M and Nassel D R, Neuropeptide signaling in insects. Adv Exp Med Biol 692: 155-165 (2010).
4. Nachman R J and Pietrantonio P V, Interaction of mimetic analogs of insect kinin neuropeptides with arthropod receptors. Adv Exp Med Biol 692: 27-48 (2010).
5. Audsley N and Down R E, G protein coupled receptors as targets for next generation pesticides. Insect Biochem Molec 67: 27-37 (2015).
6. Holman G M, Nachman R J and Coast G M, Isolation, characterization and biological activity of a diuretic myokinin neuropeptide from the housefly, Musca domestica. Peptides 20:1-10 (1999).
7. Halberg K A, Terhzaz S, Cabrero P, Davies S A and Dow J A T, Tracing the evolutionary origins of insect renal function. Nat Commun 6 (2015).
8. Schoofs L, Vanden Broeck J and De Loof A, The myotropic peptides of Locusta migratoria: structures, distribution, functions and receptors. Insect Biochem Mol Biol 23: 859-881 (1993).
9. Coast G M, Holman G M and Nachman R J, The diuretic activity of a series of cephalomyotropic neuropeptides, the achetakinins, on isolated Malpighian tubules of the house cricket Acheta domesticus. J Insect Physiol 36: 481-488 (1990).
10. Dow J A, Insights into the Malpighian tubule from functional genomics. J Exp Biol 212: 435-445 (2009).
11. Harshini S, Nachman R J and Sreekumar S, Inhibition of digestive enzyme release by neuropeptides in larvae of Opisina arenosella (Lepidoptera: Cryptophasidae). Comp Biochem Physiol B132: 353-358 (2002).
12. Nachman R J, Coast G M, Douat C, Fehrentz J A, Kaczmarek K, Zabrocki J, Pryor N W and Martinez J, A C-terminal aldehyde insect kinin analog enhances inhibition of weight gain and induces significant mortality in Helicoverpa zea larvae. Peptides 24: 1615-1621 (2003).
13. Cannell E, Doman A J, Halberg K A, Terhzaza S, Dow J A T and Davies S A, The corticotropin-releasing factor-like diuretic hormone 44 (DH44) and kinin neuropeptides modulate desiccation and starvation tolerance in Drosophila melanogaster. Peptides 80: 96-107 (2016).
14. Zandawala M, Marley R, Davies S A and Nassel D R, Characterization of a set of abdominal neuroendocrine cells that regulate stress physiology using colocalized diuretic peptides in Drosophila. Cell Mol Life Sci 75: 1099-1115 (2018).
15. Huesmann G R, Cheung C C, Loi P K, Lee T D, Swiderek K M and Tublitz N J, Amino acid sequence of CAP2b, an insect cardioacceleratory peptide from the tobacco hawkmoth Manduca sexta. FEBS Lett 371: 311-314 (1995).
16. Davies S A, Cabrero P, Povsic M, Johnston N R, Terhzaz S and Dow J A T, Signaling by drosophila capa neuropeptides. Gen Comp Endocr 188: 60-66 (2013).
17. Terhzaz S, Teets N M, Cabrero P, Henderson L, Ritchie M G, Nachman R J, Dow J A T, Denlinger D L and Davies S A, Insect capa neuropeptides impact desiccation and cold tolerance. Proc Natl Acad Sci 201501518 (2015).
18. Terhzaz S, Alford L, Yeoh J G C, Marley R, Dornan A T, Dow J A T and Davies S A, Renal neuroendocrine control of desiccation and cold tolerance by Drosophila suzukii. Pest Manag Sci 74: 800-810 (2017).
19. Predel R, Wegener C, Biology of the CAPA peptides in insects. Cell Mol Life Sci 63: 2477-2490 (2006).
20. Predel R, Nachman R J, The FXPRLamide (Pyrokinin/PBAN) Peptide Family, in Handbook of biologically active peptides, ed. by Kastin, A J, Elsevier; New York, pp. 207-212 (2006).
21. Jiang H, Wei Z, Nachman R J, Adams M E and Park Y, Functional phylogenetics reveals contributions of pleiotropic peptide action to ligand-receptor coevolution. Sci Rep 4: 6800 (2014).
22. Jiang H, Wei Z, Nachman R J, Kaczmarek K, Zabrocki J and Park Y, Functional characterization of five different PRXamide receptors of the red flour beetle Tribolium castaneum with peptidomimetics and identification of agonists and antagonists. Peptides 68: 246-252 (2015).
23. Zhang Q, Nachman R J, Kaczmarek K, Zabrocki J and Denlinger D L, Disruption of insect diapause using agonists and an antagonist of diapause hormone. Proc Natl Acad Sci USA 108: 16922-16926 (2011)
24. Cornell M J, Williams T A, Lamango N S, Coates D, Corvol P, Soubier F, Hoheisel J, Lehrach H and Isaac R E, Cloning and expression of an evolutionary conserved single-domain angiotensin converting enzyme from Drosophila melanogaster. J Biol Chem 270: 13613-13619 (1995).
25. Lamango N S, Nachman R J, Hayes T K, Strey A and Isaac R E, Hydrolysis of insect neuropeptides by an angiotensin converting enzyme from the housefly, M. domestica. Peptides 18: 47-52 (1997).
26. Nachman R J, Strey A, Isaac E, Pryor N, Lopez J D, Deng J G and Coast G M, Enhanced in vivo activity of peptidase-resistant analogs of the insect kinin neuropeptide family. Peptides 23: 735-745 (2002).
27. Nachman R J, Isaac R E, Coast G M, Roberts V A, Lange A, Orchard I, Holman G M and Favrel P, Active conformation and mimetic analog development for the Pyrokinin/PBAN and Myosuppressin insect neuropeptide families. In: Hedin P A, Hollingworth R M, Masler E P, Miyamoto J, Thompson D G (eds) Phytochemicals for pest control, ACS Symposium Series 658, ACS, Washington, D.C. pp 277-291 (1997).
28. Nachman R J, Isaac R E, Coast G M and Holman G M, Aib-containing analogues of the insect kinin neuropeptide family demonstrate resistance to an insect angiotensin-converting enzyme and potent diuretic activity. Peptides 18: 53-57 (1997).
29. Taneja-Bageshwar S, Strey A, Zubrzak P, Pietrantonio P V and Nachman R J, Comparative structure-activity analysis of insect kinin core analogs on recombinant kinin receptors from Southern cattle tick Boophilus microplus (Acari: Ixodidae) and mosquito Aedes aegypti (Diptera: Culicidae). Arch Insect Biochem Physiol 62: 128-140 (2006).
30. Taneja-Bageshwar S, Strey A, Isaac E R, Coat G M, Zubrzak P, Pietrantonio P V, Nachman R J, Biostable agonists that match or exceed activity of native insect kinins on recombinant arthropod GPCRs. Gen Comp Endocr 162: 122-128 (2009).
31. Holmes S P, He H, Chen A C, lvie G W and Pietrantonio P V, Cloning and transcriptional expression of a leucokinin-like peptide receptor from the southern cattle tick Boophilus microplus (Acari: Ixodidae). Insect Mol Biol 9: 457-465 (2000).
32. Holmes S P, Barhoumi R, Nachman R J and Pietrantonio P V, Functional analysis of a G protein-coupled receptor from the southern cattle tick Boophilus microplus (Acari: Ixodidae) identifies it as the first arthropod myokinin receptor. Insect Mol Biol 12: 27-38 (2003).
33. Pietrantonio P V, Jagge C, Taneja-Bageshwar S, Nachman R J and Barhoumi R, The mosquito Aedes aegypti (L.) leucokinin receptor is a multiligand receptor for the three Aedes kinins. Insect Mol Biol 14: 55-67 (2005).
34. Smagghe G, Mahdian K, Zubrzak P and Nachman R J, Antifeedant activity and high mortality in the pea aphid Acyrthosiphon pisum (Hemiptera: Aphidae) induced by biostable insect kinin analog. Peptides 31: 498-505 (2010).
35. Zhang C, Qu Y, Wu X, Song D, Ling Y and Yang X, Design, synthesis and aphicidal activity of N-terminal modified insect kinin analogs. Peptides 68: 233-238 (2015).
36. Radford J C, Davies S A and Dow J A, Systematic G-protein-coupled receptor analysis in Drosophila melanogaster identifies a leucokinin receptor with novel roles. J Biol Chem 277: 38810-38817 (2002).
37. Terhzaz S, Cabrero P, Robben J H, Radford J C, Hudson B D, Milligan G, Dow J A and Davies S A, Mechanism and function of Drosophila capa GPCR: a desiccation stress-responsive receptor with functional homology to human neuromedinU receptor. PLoS One 7(1): e29897 (2012).
38. Christie A E, In silico analyses of peptide paracrines/hormones in Aphidoidea. Gen Comp Endocr 159: 67-79 (2008).
39. Nault L R, Arthropod transmission of plant viruses: A new synthesis. Ann Entomol Soc Am, 90: 521-541 (1997).
40. van Emden H F and Harrington R, Aphids as Crop Pests, CABI Publishing, Wallingford, 717 p (2007).
41. Blackman R L and Eastop V F, Aphids on the World's Crops: An Identification Guide, John Wiley & Sons Ltd, Chichester, UK. (2000).
42. Dow J A T, Maddrell S H P, Gortz A, Skaer N J V, Brogan S and Kaiser K, The Malpighian tubules of Drosophila melanogaster—a novel phenotype for studies of fluid secretion and its control. J Exp Biol 197: 421-428 (1994).
43. Clough M S, Bale J S and Harrington R, Differential cold hardiness in adults and nymphs of the peach-potato aphid Myzus-Persicae. Ann Appl Biol 116: 1-9 (1990).
44. Powell S J and Bale J S, Intergenerational acclimation in aphid overwintering. Ecol Entomol 33: 95-100 (2008).
45. Sinclair B J and Chown S L, Rapid cold-hardening in a Karoo beetle, Afrinus sp. Physiol Entomol 31: 98-101 (2006).
46. Terblanche J S, Clusella-Trullas S, Deere J A and Chown S L, Thermal tolerance in a south-east African population of the tsetse fly Glossina pallidipes (Diptera, Glossinidae): Implications for forecasting climate change impacts. J Insect Physiol 54: 114-127 (2008).
47. Davies S A, Huesmann G R, Maddrell S H, O'Donnell M J, Skaer N J, Dow J A T and Tublitz N J, CAP2b, a cardioacceleratory peptide, is present in Drosophila and stimulates tubule fluid secretion via cGMP. Am J Physiol 269: R1321-R1326 (1995).
48. Douglas A E, Nutritional physiology of aphids. Adv Insect Physiol 31: 73-140 (2003).
49. Beyenbach K W, Skaer H and Dow J A, The developmental, molecular, and transport biology of Malpighian tubules. Annu Rev Entomol 55: 351-374 (2010).
50. Jing X, White T A, Yang X and Douglas A E, The molecular correlates of organ loss: The case of insect Malpighian tubules. Biol Letters 11: 20150154 (2015).
51. Sadeghi A., Van Damme, E. J. M. and Smagghe G. (2009) Evaluation of the susceptibility of the pea aphid, Acyrthosiphon pisum, to a selection of novel biorational insecticides using an artificial diet. Journal of Insect Science 9:65.
52. Van Emden F (2009). Artificial diet for aphids—thirty years' experience. REDIA, XCII: 163-167
53. For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. An insecticidal compound having the formula:
R¹-L¹-Z-R²
where Z is a peptide of formula:
L-X2-X3-F-X5-RV-
wherein:
X2 is V or Y;
X3 is A or Aib;
X5 is P or A; and
wherein optionally at least one of the residues in peptide Z is N-methylated;
L¹ is absent or is *—(C═O)C₁₋₁₀-alkylene-NH— where * denotes the point of attachment to Z;
R¹ is hydrogen, C₁₋₄ alkyl, —N⁺(R^{1a})—C(═N⁺(R^{1a})₂)NR^{1a}₂, —C(═N⁺(R^{1a})₂)NR^{1a}₂, acyl, fatty acyl, benzyl, or benzoyl; wherein each R^{1a} is independently selected from hydrogen or C₁₋₄ alkyl (e.g. methyl, ethyl, propyl, butyl); and
R² is NH₂, NR^{2a}H, NR^{2A}₂, or OR^{2a} wherein each R^{2a} if present is independently C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkylene-C₆₋₁₆-aryl, or C₁₋₆-haloalkyl, each of which may optionally be substituted with one or more groups selected from halogen, C₁₋₆-alkyl, or C₁₋₆-haloalkyl.

2. An insecticidal compound according to claim 1 wherein L¹ is absent or is *—(C═O)C₁₋₆-alkylene-NH—.

3. An insecticidal compound according to claim 2 wherein L is absent or is:

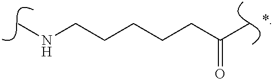

4. An insecticidal compound according to claim 1, wherein R¹ is hydrogen or —C(═N⁺(R^{1a})₂)NR^{1a}₂ such as —C(═N⁺Me₂)NMe₂.

5. An insecticidal compound according to claim 1, wherein R² is NH₂.

6. An insecticidal compound according to claim 1, wherein X2 is V and/or X5 is P.

7. An insecticidal compound according to claim 6 wherein peptide Z is:
(SEQ ID NO: 1)
LVAFPRV;
(SEQ ID NO: 2)
LVAFPR-(Me)V;
(SEQ ID NO: 3)
L-(Me)V-AFPRV;
(SEQ ID NO: 41)
LV-(Me)AFPRV
or
(SEQ ID NO: 4)
LV-(Me)A-FPR-(Me)V.

8. An insecticidal compound according to claim 7 which is:
(SEQ ID NO: 5)
H-Ahx-LVAFPRV-NH₂
(SEQ ID NO: 6)
Guanidyl-LVAFPR-(Me)V-NH₂;
(SEQ ID NO: 7)
Guanidyl-L-(Me)V-AFPRV-NH₂;
(SEQ ID NO: 9)
H-Ahx-LVAFPR-(Me)V-NH₂;
(SEQ ID NO: 10)
H-Ahx-LV-(Me)A-FPR-(Me)V-NH₂;
(SEQ ID NO: 8)
H-Ahx-L-(Me)V-AFPRV-NH₂;
(SEQ ID NO: 11)
Guanidyl-LV-(Me)A-FPRV-NH₂;
(SEQ ID NO: 12)
Palmitoyl-LVAFPRV-NH₂;
(SEQ ID NO: 13)
4-Benzoyl benzoic-LVAFPRV-NH₂;
or
(SEQ ID NO: 14)
Ac-LVAFPRV-NH₂.
wherein the terminal structure formed by “Guanidyl-L-” is:

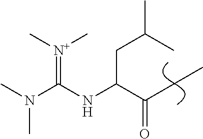 [SMILES: CCC(=O)C(CC(C)C)NC(N(C)C)=[N+](C)C]

9. An insecticidal compound according to claim 1, wherein X2 is Y and/or X5 is A.

10. An insecticidal compound according to claim 9 wherein peptide Z is:
(SEQ ID NO: 15)
LYAFARV;
(SEQ ID NO: 16)
LYAFAR-(Me)V;
(SEQ ID NO: 17)
LYAF-(Me)A-RV;
(SEQ ID NO: 18)
(Me)L-YAFARV;
or
(SEQ ID NO: 19)
LY-Aib-FARV.

11. An insecticidal compound according to claim 9 wherein L¹ is absent.

12. An insecticidal compound according to claim 9, wherein R¹ is H.

13. An insecticidal compound according to claim 9, wherein R² is NH₂.

14. An insecticidal compound according to claim 9, which is:
(SEQ ID NO: 20)
H-LYAFARV-NH₂;
(SEQ ID NO: 21)
H-LYAFAR-(Me)V-NH₂;
(SEQ ID NO: 23)
H-LYAF-(Me)A-RV-NH₂;
(SEQ ID NO: 24)
H-(Me)L-YAFARV-HN₂;
(SEQ ID NO: 25)
H-LY-Aib-FARV-NH₂;
(SEQ ID NO: 22)
H-LY-(Me)A-FARV-NH₂;
(SEQ ID NO: 26)
4-Benzoyl benzoic-LYAFPRV-NH₂;
(SEQ ID NO: 27)
Ac-LYAFPRV-NH₂;
(SEQ ID NO: 28)
Palmitoyl-LYAFPRV-NH₂;
or
(SEQ ID NO: 29)
H-Nle-LYAFPRV-NH₂.

15. An insecticidal composition, comprising a compound according to claim 1 in admixture with one or more solvents, carriers, diluents, adjuvants, preservatives, dispersants, emulsifying agents, or synergists.

16. An insecticidal composition according to claim 15 which is an aqueous composition.

17-20. (canceled)

21. A method of reducing or inhibiting infestation of a plant by hemipteran and/or dipteran insects, or of reducing hemipteran and/or dipteran insect load on a plant comprising contacting the plant with an insecticidal compound as described in claim 1.

22. A method according to claim 22 wherein the compound is applied to the plant while the plant is free or substantially free of hemipteran and/or dipteran insects.

23. (canceled)

24. An insecticidal composition according to claim 15 wherein the composition is an insect control composition or a plant protection composition.


SEQUENCE LISTING (41 sequences)

SEQ ID NO: 1 - protein, 7 aa, Artificial Sequence
  peptide Z example
LVAFPRV

SEQ ID NO: 2 - protein, 7 aa, Artificial Sequence
  peptide Z example
  MOD_RES (7)..(7): MeVal
LVAFPRV

SEQ ID NO: 3 - protein, 7 aa, Artificial Sequence
  peptide Z example
  MOD_RES (2)..(2): MeVal
LVAFPRV

SEQ ID NO: 4 - protein, 7 aa, Artificial Sequence
  peptide Z example
  MOD_RES (3)..(3): N-methylalanine
  MOD_RES (7)..(7): MeVal
LVAFPRV

SEQ ID NO: 5 - protein, 8 aa, Artificial Sequence
  AH56
  MOD_RES (1)..(1): 6-aminohexanoic acid
  MOD_RES (8)..(8): AMIDATION
XLVAFPRV

SEQ ID NO: 6 - protein, 7 aa, Artificial Sequence
  AH257
  MISC_FEATURE (1)..(1): Guanidyl
  MOD_RES (7)..(7): MeVal
  MOD_RES (7)..(7): AMIDATION
LVAFPRV

SEQ ID NO: 7 - protein, 7 aa, Artificial Sequence
  AH259
  MISC_FEATURE (1)..(1): Guanidyl
  MOD_RES (2)..(2): MeVal
  MOD_RES (7)..(7): AMIDATION
LVAFPRV

SEQ ID NO: 8 - protein, 8 aa, Artificial Sequence
  AH283
  MISC_FEATURE (1)..(1): 6-aminohexanoic acid
  MOD_RES (3)..(3): MeVal
  MOD_RES (8)..(8): AMIDATION
XLVAFPRV

SEQ ID NO: 9 - protein, 8 aa, Artificial Sequence
  AH283a
  MISC_FEATURE (1)..(1): 6-aminohexanoic acid
  MOD_RES (8)..(8): MeVal
  MOD_RES (8)..(8): AMIDATION
XLVAFPRV

SEQ ID NO: 10 - protein, 8 aa, Artificial Sequence
  AH270
  MOD_RES (1)..(1): 6-aminohexanoic acid
  MOD_RES (4)..(4): N-methylalanine
  MOD_RES (8)..(8): MeVal
  MOD_RES (8)..(8): AMIDATION
XLVAFPRV

SEQ ID NO: 11 - protein, 7 aa, Artificial Sequence
  AH258
  MISC_FEATURE (1)..(1): Guanidyl
  MOD_RES (3)..(3): N-methylalanine
  MOD_RES (7)..(7): AMIDATION
LVAFPRV

SEQ ID NO: 12 - protein, 7 aa, Artificial Sequence
  AH59
  MISC_FEATURE (1)..(1): Palmitoyl
  MOD_RES (7)..(7): AMIDATION
LVAFPRV

SEQ ID NO: 13 - protein, 7 aa, Artificial Sequence
  AH55
  MISC_FEATURE (1)..(1): 4-Benzoyl benzoic
  MOD_RES (7)..(7): AMIDATION
LVAFPRV

SEQ ID NO: 14 - protein, 7 aa, Artificial Sequence
  AH33
  MOD_RES (1)..(1): ACETYLATION
  MOD_RES (7)..(7): AMIDATION
LVAFPRV

SEQ ID NO: 15 - protein, 7 aa, Artificial Sequence
  Peptide Z example
LYAFARV

SEQ ID NO: 16 - protein, 7 aa, Artificial Sequence
  Peptide Z example
  MOD_RES (7)..(7): MeVal
LYAFARV

SEQ ID NO: 17 - protein, 7 aa, Artificial Sequence
  Peptide Z example
  MOD_RES (5)..(5): N-methylalanine
LYAFARV

SEQ ID NO: 18 - protein, 7 aa, Artificial Sequence
  Peptide Z example
  MOD_RES (1)..(1): N-methylleucine
LYAFARV

SEQ ID NO: 19 - protein, 7 aa, Artificial Sequence
  Peptide Z example
  MOD_RES (3)..(3): Aib
LYXFARV

SEQ ID NO: 20 - protein, 7 aa, Artificial Sequence
  AH188
  MOD_RES (7)..(7): AMIDATION
LYAFARV

SEQ ID NO: 21 - protein, 7 aa, Artificial Sequence
  AH380
  MOD_RES (7)..(7): MeVal
  MOD_RES (7)..(7): AMIDATION
LYAFARV

SEQ ID NO: 22 - protein, 7 aa, Artificial Sequence
  AH382
  MOD_RES (3)..(3): N-methylalanine
  MOD_RES (7)..(7): AMIDATION
LYAFARV

SEQ ID NO: 23 - protein, 7 aa, Artificial Sequence
  AH382a
  MOD_RES (5)..(5): N-methylalanine
  MOD_RES (7)..(7): AMIDATION
LYAFARV

SEQ ID NO: 24 - protein, 7 aa, Artificial Sequence
  AH383
  MOD_RES (1)..(1): N-methylleucine
  MOD_RES (7)..(7): AMIDATION
LYAFARV

SEQ ID NO: 25 - protein, 7 aa, Artificial Sequence
  AH387
  MOD_RES (3)..(3): Aib
  MOD_RES (7)..(7): AMIDATION
LYXFARV

SEQ ID NO: 26 - protein, 7 aa, Artificial Sequence
  AH49
  MISC_FEATURE (1)..(1): 4-Benzoyl benzoic
  MOD_RES (7)..(7): AMIDATION
LYAFPRV

SEQ ID NO: 27 - protein, 7 aa, Artificial Sequence
  AH32
  MOD_RES (1)..(1): ACETYLATION
  MOD_RES (7)..(7): AMIDATION
LYAFPRV

SEQ ID NO: 28 - protein, 7 aa, Artificial Sequence
  AH62
  MISC_FEATURE (1)..(1): Palmitoyl
  MOD_RES (7)..(7): AMIDATION
LYAFPRV

SEQ ID NO: 29 - protein, 8 aa, Artificial Sequence
  AH51
  MOD_RES (1)..(1): Nle
  MOD_RES (8)..(8): AMIDATION
XLYAFPRV

SEQ ID NO: 30 - protein, 8 aa, Artificial Sequence
  PEGAH270/AHPEG270
  MOD_RES (1)..(1): 6-aminohexanoic acid
  MOD_RES (1)..(1): pegulated
  MOD_RES (4)..(4): N-methylalanine
  MOD_RES (8)..(8): MeVal
  MOD_RES (8)..(8): AMIDATION
XLVAFPRV

SEQ ID NO: 31 - protein, 12 aa, Artificial Sequence
  CAPA1
  MOD_RES (12)..(12): AMIDATION
GANMGLYAFPRV

SEQ ID NO: 32 - protein, 10 aa, Artificial Sequence
  CAPA2
  MOD_RES (10)..(10): AMIDATION
ASGLVAFPRV

SEQ ID NO: 33 - protein, 12 aa, Artificial Sequence
  Myzpe-CAPA-1
  MOD_RES (12)..(12): AMIDATION
ESVAGLIPFPRV

SEQ ID NO: 34 - protein, 9 aa, Artificial Sequence
  Myzpe-CAPA-2
  MOD_RES (9)..(9): AMIDATION
EGLIPFPRI

SEQ ID NO: 35 - protein, 14 aa, Artificial Sequence
  Myzpe-CAPA-3
  MOD_RES (14)..(14): AMIDATION
SDSKNTALWFGPRL

SEQ ID NO: 36 - protein, 10 aa, Artificial Sequence
  Apime-CAPA-2
  MOD_RES (10)..(10): AMIDATION
AFGLLTYPRI

SEQ ID NO: 37 - protein, 10 aa, Artificial Sequence
  Bomte-CAPA
  MOD_RES (10)..(10): AMIDATION
ASGLMAYPRV

SEQ ID NO: 38 - protein, 12 aa, Artificial Sequence
  Drosu-CAPA-1
  MOD_RES (12)..(12): AMIDATION
GANMGLYAFPRV

SEQ ID NO: 39 - protein, 10 aa, Artificial Sequence
  Drosu-CAPA-2
  MOD_RES (10)..(10): AMIDATION
ASGLVAFPRV

SEQ ID NO: 40 - protein, 7 aa, Artificial Sequence
  minimal heptameric pharmacophore of the Capa peptide
LYAFPRV

SEQ ID NO: 41 - protein, 7 aa, Artificial Sequence
  peptide Z example
  MOD_RES (3)..(3): N-methylalanine
LVAFPRV